(19) United States
(12) Patent Application Publication
(10) Pub. No.: US 2023/0000903 A1
(43) Pub. Date: Jan. 5, 2023

(54) METHODS AND SYSTEMS FOR NITRIC OXIDE THERAPY FOR THE TREATMENT OR PREVENTION OF RESPIRATORY INFECTIONS

(71) Applicant: KNOW Bio, LLC, Durham, NC (US)

(72) Inventors: Nathan Stasko, Durham, NC (US); Kyle Kimble, Durham, NC (US)

(21) Appl. No.: 17/929,511

(22) Filed: Sep. 2, 2022

Related U.S. Application Data
(63) Continuation-in-part of application No. PCT/US21/20725, filed on Mar. 3, 2021.
(60) Provisional application No. 62/990,832, filed on Mar. 17, 2020.
(60) Provisional application No. 62/985,869, filed on Mar. 5, 2020.

(51) Int. Cl. A61K 33/00; A61P 11/00

(52) U.S. Cl. CPC A61K 33/00; A61P 11/00

(57) ABSTRACT

Methods for the treatment of a respiratory infection, for the prevention of worsening of symptoms associated with the infection, and for reducing the lethality of the infection such as but not limited to respiratory infections caused by a coronavirus. The present disclosure provides specific gaseous nitric oxide (NO) dosing regimens optionally paired with the monitoring of toxicology outcomes so as to enable the use of effective NO doses for treatment purposes. The present invention also discloses air circulation systems featuring NO for helping to prevent respiratory infections.

### CROSS-REFERENCES TO RELATED APPLICATIONS

This application is a continuation-in-part, and claims benefit of PCT Application No. PCT/US2021/020725 filed Mar. 3, 2021, which claims benefit of U.S. Provisional Patent Application No. 62/985,869 filed Mar. 5, 2020, and U.S. Provisional Patent Application No. 62/990,832 filed Mar. 17, 2020, the specifications of which are incorporated herein in their entirety by reference.

### BACKGROUND OF THE INVENTION

### Field of the Invention

The present invention relates to methods for treating infections such as respiratory infections by administration of gaseous nitric oxide, methods for preventing said infections, and gaseous nitric oxide (NO) regimens. The present invention also relates to air circulation systems, both large and small scale, featuring gaseous nitric oxide.

### Background Art

2019-new Coronavirus (2019-nCoV) infection (COVID-19) is highly contagious and responsible for thousands of casualties world-wide. Because it is highly contagious, efforts to prevent its spread appear to be largely ineffective. The progression of infection begins with an extended period of mild symptoms typical of a viral respiratory infection. However, approximately 20% of patients with the viral infection develop Severe Acute Respiratory Syndrome (SARS), accompanied with acute lung injury or acute respiratory distress syndrome. Patients succumbing to this infection all experience this progression to SARS. While therapies which are efficacious against SARS (or the related Cytokine Release Syndrome (CRS) that it initiates), a method of treating a patient with a therapeutic to prevent the development of SARS is highly desirable. There is an urgent need for a broadly deployed therapeutic effective against the development of SARS from a coronavirus infection.

Gaseous nitric oxide (NO) has been tried as a means of treating a number of respiratory indications, but often with little success because it has been difficult to determine correct dosing schedules and reconcile dosing with toxicology outcomes to enable the use of high (and effective) doses.

From the toxicological aspect, NO has a half-life in the body of less than 6 seconds and a radius of action of approximately 200 microns from its site of origin, beyond which it is inactivated through binding to sulfhydryl groups of cellular thiols or by nitrosylation of the heme moieties of hemoglobin to form methemoglobin (MetHb). MetHb reductase reduces NO to nitrates in the blood serum. Nitrate has been identified as the predominant nitric oxide metabolite excreted in the urine, accounting for more than 70% of the nitric oxide dose inhaled. Nitrate is cleared from the plasma by the kidney at rates approaching the rate of glomerular filtration. Blood levels of MetHb in healthy humans are typically less than 2%.

Potential side effects of high dose NO treatment hence include the binding of NO to hemoglobin and the formation of MetHb, which could lead to decreased oxygen transport, and the capacity of NO to act as a nitrosylating agent on proteins and other cell constituents. Acute pulmonary injury, pulmonary edema, hemorrhage, changes in surface tension of surfactant, reduced alveolar numbers, and airway responsiveness are associated with high airway levels of NO, NO₂, and other oxides of nitrogen. Thus, concerns have been raised regarding the potential use of NO as a therapeutic agent in various clinical scenarios.

Without wishing to limit the present invention to any theory or mechanism, it is believed that the methods of the present invention can help prevent the coronavirus infection from developing into SARS. Notably, certain prior art methods that have attempted treatment of coronavirus with NO therapy have not been very effective. Such methods have focused on patients already experiencing SARS due to their coronavirus infection. At this stage of infection, the major complication is the Cytokine Release Syndrome (CRS) caused by the viral infection rather than the virus itself. While stopping further infection at this point is expected to be beneficial, it may not be sufficient to solve that patient's current problem, which is the CRS. Indeed, the prior art reflects that NO (at currently accepted administered doses) has been evaluated for ARDS and found to be not sufficiently effective. In addition, prior art methods use low levels of NO gas, which may not be sufficient.

### BRIEF SUMMARY OF THE INVENTION

Certain aspects of the disclosure relate to methods for the treatment of an infection, for the prevention of worsening of symptoms associated with the infection, and for ameliorating symptoms associated with the infection. Certain aspects of the disclosure relate to methods for prevention of an infection. Certain aspects of the disclosure relate to methods of inhibiting proliferation of a virus in a lung of a patient. Certain aspects of the disclosure relate to methods for reducing the lethality of an infection (e.g., reduction in lethality of a coronavirus). Certain aspects of the disclosure relate to methods for preventing severe acute respiratory syndrome (SARS).

Infections may include but are not limited to those caused by a coronavirus. Non-limiting examples of human coronaviruses include SARS-CoV, SARS-CoV-2, MERS-CoV, HCoV-229E, HCoV-NL63, HCoV-OC43, HCoV-HKU1, etc. The present invention is not limited to coronavirus infections such as COVID-19 caused by SARS-CoV-2.

The methods herein may be used to treat a patient with an infection, e.g., a coronavirus infection, at any particular stage of infection. However, the present invention provides early intervention methods, e.g., methods for treating patients early in the progression of the disease so as to help avoid severe respiratory injury, e.g., to help prevent the development of SARS.

The present disclosure provides specific gaseous nitric oxide (NO) dosing regimens optionally paired with the monitoring of toxicology outcomes so as to enable the use of effective NO doses for treatment purposes.

As a non-limiting example, in certain embodiments, a patient with an infection (e.g., coronavirus infection) (but without SARS) is treated for 30 minutes with a NO concentration of 300 ppm, and then a dose of 80 ppm for 2 hours. SpMet may be monitored, and if it exceeds a certain threshold (e.g., 3%, 4%, 5%, 6%, 7%, 8%, 9%, 10%, 15%, 20%, etc.), NO administration may be discontinued. If symptoms worsen, the therapy may be repeated, e.g., whenever SpMet drops below a certain threshold, e.g., 5%.

In another aspect, the disclosure relates to feedback loop methods for treating an infection, for the prevention of worsening of symptoms associated with the infection, for ameliorating symptoms associated with the infection, for prevention of an infection, for the prevention of SARS, etc. For example, the method comprises measuring certain parameters in a patient and using the results of the measurements of the parameters to determine (and administer) an appropriate treatment to administer to the patient in order to achieve desired results of the measurements of the parameters (or variables).

Non-limiting examples of parameters (or variables) that may be measured and/or analyzed include: methemoglobin (metHB) level (SpMet), a blood nitrate level, an oxygen saturation level (SpO₂), heart rate, respiration rate, body temperature, blood pressure, an inflammatory cytokine plasma level, a proinflammatory cytokine serum level, a C-reactive protein (CRP) level, a level of lymphocytes, an erythrocyte sedimentation rate (ESR), a procalcitonin (PCT) level, or a serum amyloid A (SAA) protein level, etc.

As an example, the methods herein may include a feedback loop method for treating a coronavirus infection, preventing worsening of symptoms of the coronavirus infection, or ameliorating symptoms of the coronavirus infection in a patient in need thereof, wherein the method comprises (a) determining an observed state of a set of variables in the patient, the set of variables comprises one or a combination of: a methemoglobin (metHB) level (SpMet), a blood nitrate level, an oxygen saturation level (SpO₂), heart rate, respiration rate, body temperature, blood pressure, an inflammatory cytokine plasma level, a proinflammatory cytokine serum level, a C-reactive protein (CRP) level, a level of lymphocytes, an erythrocyte sedimentation rate (ESR), a procalcitonin (PCT) level, or a serum amyloid A (SAA) protein level; (b) choosing an appropriate treatment to administer to the patient in order to achieve a target state of the set of variables, the appropriate treatment is chosen based on the observed state of the set of variables in (a); and (c) administering the appropriate treatment decided upon in (b) to the patient in order to achieve the target state of the set of variables. The method may continuously repeat (a), (b), and (c) in order in a loop. In some embodiments, the appropriate treatment comprises administering to the patient at least one nitric oxide (NO) treatment, the NO treatment comprising a dose of NO for a time period. In some embodiments, the appropriate treatment further comprises administering at least one oxygen treatment subsequent to the at least one NO treatment, the oxygen treatment comprising a dose of oxygen for a time period, the oxygen treatment is essentially free of NO. In some embodiments, the appropriate treatment comprises administering to the patient at least one nitric oxide (NO) treatment, the NO treatment comprising a dose of NO for a time period, and after completion of the at least one NO treatment subsequently administering at least one oxygen treatment, the oxygen treatment comprising a dose of oxygen for a time period, the oxygen treatment is essentially free of NO.

The present invention also features a method of treating a coronavirus infection or suspected coronavirus infection in a patient, preventing worsening of symptoms associated with the coronavirus infection or suspected coronavirus infection, or ameliorating symptoms associated with the coronavirus infection or suspected coronavirus infection, wherein the method comprises (a) administering to the patient after exposure or suspected exposure to the coronavirus at least one nitric oxide (NO) treatment, the NO treatment comprising a dose of NO for a time period; followed by (b) administering at least one oxygen treatment after cessation of the at least one NO treatment, the oxygen treatment comprising a dose of oxygen for a time period, the oxygen treatment is essentially free of NO; wherein (a) and (b) are performed as one cycle or repeated for at least one additional cycle. The oxygen treatment may be effective for reducing interaction between oxygen and NO that otherwise creates harmful NOx species. The NO treatment may be effective for inhibiting replication of the coronavirus, preventing worsening of symptoms associated with the coronavirus, or ameliorating symptoms associated with the coronavirus. In some embodiments, the method further comprises, before step (a), after step (a), after step (b), or a combination thereof, (i) determining an observed state of a set of variables in the patient, the set of variables comprises one or a combination of: a methemoglobin (metHB) level (SpMet), a blood nitrate level, an oxygen saturation level (SpO₂), heart rate, respiration rate, body temperature, blood pressure, an inflammatory cytokine plasma level, a proinflammatory cytokine serum level, a C-reactive protein (CRP) level, a level of lymphocytes, an erythrocyte sedimentation rate (ESR), a procalcitonin (PCT) level, or a serum amyloid A (SAA) protein level; (ii) choosing an appropriate treatment to administer to the patient in order to achieve a target state of the set of variables, the appropriate treatment is chosen based on the observed state of the set of variables in (i); and (iii) administering the appropriate treatment decided upon in (ii) to the patient in order to achieve the target state of the set of variables; wherein the method continuously repeats (i), (ii), and (iii) in order in a loop, wherein the target state of the set of variables is for treating a coronavirus infection, preventing worsening of symptoms thereof, or ameliorating symptoms thereof. In some embodiments, the appropriate treatment comprises administering to the patient at least one nitric oxide (NO) treatment, the NO treatment comprising a dose of NO for a time period. In some embodiments, the appropriate treatment further comprises administering at least one oxygen treatment subsequent to the at least one NO treatment, the oxygen treatment comprising a dose of oxygen for a time period, the oxygen treatment is essentially free of NO. In some embodiments, the appropriate treatment comprises administering to the patient at least one nitric oxide (NO) treatment, the NO treatment comprising a dose of NO for a time period, and after completion of the at least one NO treatment subsequently administering at least one oxygen treatment, the oxygen treatment comprising a dose of oxygen for a time period, the oxygen treatment is essentially free of NO.

In another aspect, the disclosure relates to providing specific gaseous nitric oxide (NO) dosing regimens in air circulating systems, e.g., for the purpose of preventing infections, for the purpose of reducing spread of infections, etc. Air circulating systems may include but are not limited to large capacity systems, e.g., systems in spaces equipped to house, shelter, transport, or enclose large numbers of people (e.g., trains, airplanes, buildings such as apartment complexes, offices, shopping locations, etc.), and smaller capacity systems, e.g., systems in spaces equipped to house, shelter, transport or enclose small numbers of people (e.g., passenger cars, trucks, single-family homes, small stores, etc.).

The present invention provides feedback loop methods for treating an infection (e.g., coronavirus infection or other infection as described herein), preventing worsening of symptoms of the infection, or ameliorating symptoms of the infection in a patient in need thereof. In some embodiments, the method comprises determining an observed state of a set of variables in the patient (e.g., measuring a set of parameters), the set of variables comprises one or a combination of: a methemoglobin (metHB) level (SpMet), a blood nitrate level, an oxygen saturation level (SpO₂), heart rate, respiration rate, body temperature, blood pressure, an inflammatory cytokine plasma level, a proinflammatory cytokine serum level, a C-reactive protein (CRP) level, a level of lymphocytes, an erythrocyte sedimentation rate (ESR), a procalcitonin (PCT) level, or a serum amyloid A (SAA) protein level. The method further comprises choosing an appropriate treatment to administer to the patient in order to achieve a target state of the set of variables (e.g., the appropriate treatment is chosen based on the observed state of the set of variables). The method further comprises administering the appropriate treatment decided upon to the patient in order to achieve the target state of the set of variables. The method may comprise continuously repeating the above steps in order in a loop. In certain embodiments, the target state of the set of variables (e.g., the desired result of the measurements of the parameters) is for treating an infection (e.g., coronavirus infection), preventing worsening of symptoms thereof, or ameliorating symptoms thereof.

In some embodiments, the patient does not have Severe Acute Respiratory Syndrome (SARS).

In some embodiments, the appropriate treatment is a dose of 500 ppm nitric oxide (NO) for a time frame from 5 to 30 minutes. In some embodiments, the appropriate treatment is a dose of 300 ppm nitric oxide (NO) for a time frame from 5 to 45 minutes. In some embodiments, the appropriate treatment is a dose of 100 ppm nitric oxide (NO) for a time frame from 1 to 4 hours. In some embodiments, the appropriate treatment is a dose of 80 ppm nitric oxide (NO) for a time frame from 1 to 4 hours. In some embodiments, the appropriate treatment is a dose of 300 ppm NO for a time frame of 30 minutes followed by a dose of 80 ppm NO for a time frame of 2 hours. In some embodiments, the appropriate treatment is a dose of 200 ppm NO for a time frame of 10 minutes followed by a dose of 80 ppm NO for a time frame of 60 minutes. In some embodiments, the appropriate treatment is a dose of 50 ppm nitric oxide (NO) for a time frame of at least 5 hours.

In some embodiments, the appropriate treatment is a maintenance dose of NO. In some embodiments, the maintenance dose of NO is a time weighted average of 36 ppm NO administered over a time frame from 10 to 12 hours. In some embodiments, the maintenance dose of NO is a time weighted average of 72 ppm NO administered over a time frame from 10 to 12 hours. In some embodiments, the maintenance dose of NO is a time weighted average of from 36 to 360 ppm NO administered over a time frame from 4 to 12 hours.

In some embodiments, the appropriate treatment is a single dose of NO. In some embodiments, the appropriate treatment is a combination of doses of NO.

In some embodiments, the target state of the set of variables is a SpMet from 2-20%. In some embodiments, the target state of the set of variables is a SpMet from 3-10%. In some embodiments, the target state of the set of variables is a SpMet from 5-15%. In some embodiments, the target state of the set of variables is a SpMet of at least 5%. In some embodiments, determining the observed state of the set of variables comprises measuring SpMet using a non-invasive mechanism. In some embodiments, determining the observed state of the set of variables comprises measuring SpMet using a pulse CO-oximeter. In some embodiments, the target state of the set of variables is a blood nitrate level from 2,000 ng/mL to 15,000 ng/mL. In some embodiments, the target state of the set of variables is a blood nitrate level of 1,400 ng/mL to 15,000 ng/mL.

In some embodiments, the proinflammatory cytokine is IL-1b, IFN-gamma, IP-10, or MCP-1.

In some embodiments, the method is repeated in the loop at least 2× in a 24 hour period. In some embodiments, the method is repeated in the loop at least 4× in a 24 hour period. In some embodiments, the method is repeated in the loop at least 5× in a 24 hour period. In some embodiments, the appropriate treatment is different for each repeat of the loop. In some embodiments, the appropriate treatment is the same for each repeat of the loop. In some embodiments, a portion of the repeats of the loop have the same appropriate treatment and a portion of the repeats of the loop have a different appropriate treatment. In some embodiments, the method is repeated in the loop over a 3 day period. In some embodiments, the method is repeated in the loop over a 5 day period. In some embodiments, the method is repeated in the loop over a 7 day period.

The method may inhibit replication of the infectious agent, e.g., coronavirus. In some embodiments, the infectious agent (e.g., coronavirus) is undetectable 7 days after administering the at least one NO treatment to the patient. In some embodiments, the infectious agent (e.g., coronavirus) is undetectable 14 days after administering the at least one NO treatment to the patient. In some embodiments, the infectious agent (e.g., coronavirus) is undetectable 28 days after administering the at least one NO treatment to the patient.

In some embodiments, the method further comprises administering a compound that increases levels of methemoglobin reductase. In some embodiments, the method further comprises administering an immunosuppressant.

In some embodiments, the method prevents Severe Acute Respiratory Syndrome (SARS). In some embodiments, the method prevents the patient from having a SpO₂<93% without oxygen supplementation sustained for more than 12 hours. In some embodiments, the method prevents the patient from having a PaO2/FiO2 ratio <300 mmHg sustained for more than 12 hours. In some embodiments, the method prevents the patient from needing high flow nasal cannula oxygen, or intubation and mechanical ventilation, or ECMO therapy over 7 or more days.

In some embodiments, the NO treatment comprises a breath loaded with NO, wherein the NO is at a concentration from 20-500 ppm.

In some embodiments, the breath further comprises oxygen at a concentration from 5-100%. In some embodiments, the breath further comprises oxygen at a concentration from 5-20%. In some embodiments, the oxygen treatment comprises a breath loaded with oxygen, the oxygen is at a concentration from 2-100%. In some embodiments, the oxygen is balanced with N₂, ambient air, another gas, or a combination thereof.

In some embodiments, the NO treatment is delivered to the mouth of the patient, and the oxygen treatment is delivered to the nose of the patient. In some embodiments, the NO treatment is delivered to the nose of the patient, and the oxygen treatment is delivered to the mouth of the patient.

In some embodiments, the method further comprises co-administering a secondary therapeutic agent. In some embodiments, the secondary therapeutic agent is an antiviral drug. Non-limiting examples of antiviral drugs include remdesivir, favipiravir, lopinavir/ritonavir, duranavir/cobicistat, and umifenovir (Arbidol). In some embodiments, the secondary therapeutic agent is chloroquine or amodiquine. In some embodiments, the secondary therapeutic agent is bevacizumab. In some embodiments, the secondary therapeutic agent is alpha-interferon. In some embodiments, the secondary therapeutic agent is a corticosteroid (e.g., methylprednisolone, dexamethasone, the like, or a combination thereof). In some embodiments, the secondary therapeutic agent is an antibody. In some embodiments, the secondary therapeutic agent is administered via inhalation. In some embodiments, the secondary therapeutic agent is administered systemically.

In some embodiments, the dose of NO comprises NO and a carrier gas. In some embodiments, the carrier gas comprises a blend of nitric oxide and nitrogen. The blend of nitric oxide and nitrogen may be delivered as INOmax. In some embodiments, the carrier gas is argon. In some embodiments, the concentration of argon is at least 2%. In some embodiments, the carrier gas is helium. Helium may be delivered as Heliox. In some embodiments, the carrier gas is carbon dioxide. In some embodiments, the carrier gas is carbon monoxide. In some embodiments, the carbon monoxide is delivered at a level up to 1000 ppm.

In some embodiments, the method further comprises administering a NO gas transmission facilitator. NO gas transmission facilitators are known in the art and include but are not limited to perfluoron, fluosol, a perfluorinated hydrocarbon, and a pulmonary surfactant. In some embodiments, the pulmonary surfactant is dipalmitoylphosphatidylcholine (DPPC), phosphatidylcholine, a surfactant protein, colfosceril palmitate, pumactant, KL-4, venticute, lucinactant, beractant, calfactant, poractant alfa, or a combination thereof. In some embodiments, the NO gas transmission facilitator is administered via inhalation.

In some embodiments, the method further comprises integrating a NOx scrubbing filter into a gas delivery tube used for delivering the dose of NO.

The present invention also provides a method of treating an infection (e.g., coronavirus infection) or suspected infection (e.g., suspected coronavirus infection) in a patient, preventing worsening of symptoms associated with the infection or suspected infection, or ameliorating symptoms associated with the infection or suspected infection. In some embodiments, the method comprises administering to the patient after exposure or suspected exposure to the infectious agent (e.g., coronavirus) at least one nitric oxide (NO) treatment (e.g., a dose of NO for/over a time period), wherein the NO treatment is effective for inhibiting replication of the infectious agent (e.g., coronavirus), preventing worsening of symptoms associated with the infectious agent (e.g., coronavirus), or ameliorating symptoms associated with the infectious agent (e.g., coronavirus).

In some embodiments, the patient does not have Severe Acute Respiratory Syndrome (SARS).

In some embodiments, the at least one NO treatment is a dose of at least 160 ppm NO for a time period of 30 minutes. In some embodiments, the at least one NO treatment is a dose of 500 ppm NO for a time period from 5 to 30 minutes. In some embodiments, the at least one NO treatment is a dose of 300 ppm NO for a time period from 5 to 45 minutes. In some embodiments, the at least one NO treatment is a dose of 100 ppm NO for a time period from 1 to 4 hours. In some embodiments, the at least one NO treatment is a dose of 80 ppm NO for a time period from 1 to 4 hours. In some embodiments, the at least one NO treatment is a dose of 300 ppm NO for a time period of 30 minutes, followed by a dose of 80 ppm NO for a time period of 2 hours. In some embodiments, the at least one NO treatment is a dose of 200 ppm NO for a time period of 10 minutes, followed by a dose of 80 ppm NO for a time period of 60 minutes. In some embodiments, the at least one NO treatment is a dose of 50 ppm NO for a time period of at least 5 hours.

In some embodiments, the at least one NO treatment is a maintenance dose. In some embodiments, the maintenance dose of NO is a time weighted average of 36 ppm NO administered over a time frame from 10 to 12 hours. In some embodiments, the maintenance dose of NO is a time weighted average of 72 ppm NO administered over a time frame from 10 to 12 hours. In some embodiments, the maintenance dose of NO is a time weighted average of from 36 to 360 ppm NO administered over a time frame from 4 to 12 hours.

In some embodiments, the at least one NO treatment is a single dose of NO. In some embodiments, the at least one NO treatment is a combination of doses of NO. In some embodiments, the at least one NO treatment is repeated 2 or more times over a 24 hour period. In some embodiments, the at least one NO treatment is repeated 4 or more times over a 24 hour period. In some embodiments, the at least one NO treatment is repeated 5 or more times over a 24 hour period. In some embodiments, the at least one NO treatment is repeated 2 or more times over a 2 day period. In some embodiments, the at least one NO treatment is repeated 2 or more times over a 5 day period. In some embodiments, the at least one NO treatment is repeated 5 or more times over a 2 day period. In some embodiments, the at least one NO treatment is repeated 5 or more times over a 5 day period. In some embodiments, the at least one NO treatment is repeated 25 or more times over a 5 day period.

In some embodiments, the infectious agent (e.g., coronavirus) is undetectable 7 days after administering the at least one NO treatment to the patient. In some embodiments, the infectious agent (e.g., coronavirus) is undetectable 14 days after administering the at least one NO treatment to the patient. In some embodiments, the infectious agent (e.g., coronavirus) is undetectable 28 days after administering the at least one NO treatment to the patient.

In some embodiments, the method further comprises measuring metHb levels (SpMet) in a sample from the patient. In some embodiments, the at least one NO treatment is administered such that the SpMet is from 2-20%. In some embodiments, the at least one NO treatment is administered such that the SpMet is from 3-10%. In some embodiments, the at least one NO treatment is administered such that the SpMet is from 5-15%. In some embodiments, the at least one NO treatment is administered such that the SpMet is at least 5%. In some embodiments, SpMet is measured using a non-invasive mechanism, e.g., a pulse CO-oximeter.

In some embodiments, the method further comprises measuring blood nitrate levels in a sample from the patient. In some embodiments, the at least one NO treatment is administered such that the blood nitrate level is from 2,000 ng/mL to 15,000 ng/mL. In some embodiments, the at least one NO treatment is administered such that the blood nitrate level is from 1,400 ng/mL to 15,000 ng/mL. In some embodiments, if the blood nitrate level is over 15,000 ng/mL, then the administration of the at least one NO treatment is stopped.

In some embodiments, the method further comprises administering a compound that increases levels of methemoglobin reductase. In some embodiments, the method further comprises administering an immunosuppressant.

In some embodiments, the method further comprises measuring in the patient one or a combination of: an oxygen saturation level (SpO₂), heart rate, respiration rate, body temperature, blood pressure, an inflammatory cytokine plasma level, a proinflammatory cytokine serum level (e.g., IL-1 b, IFN-gamma, IP-10, or MCP-1), C-reactive protein (CRP) level, a level of lymphocytes, an erythrocyte sedimentation rate (ESR), a procalcitonin (PCT) level, or a serum amyloid A (SAA) protein level.

In some embodiments, the method prevents Severe Acute Respiratory Syndrome (SARS). In some embodiments, the method prevents the patient from having a SpO₂<93% without oxygen supplementation sustained for more than 12 hours. In some embodiments, the method prevents the patient from having a PaO2/FiO2 ratio <300 mmHg sustained for more than 12 hours. In some embodiments, the method prevents the patient from needing high flow nasal cannula oxygen, or intubation and mechanical ventilation, or ECMO therapy over 7 or more days.

In some embodiments, the NO treatment comprises a breath loaded with NO, wherein the NO is at a concentration from 20-500 ppm.

In some embodiments, the breath further comprises oxygen at a concentration from 5-100%. In some embodiments, the breath further comprises oxygen at a concentration from 5-20%. In some embodiments, the oxygen treatment comprises a breath loaded with oxygen, the oxygen is at a concentration from 2-100%. In some embodiments, the oxygen is balanced with N₂, ambient air, another gas, or a combination thereof.

In some embodiments, the NO treatment is delivered to a mouth of the patient and the oxygen treatment is delivered to a nose of the patient. In some embodiments, the NO treatment is delivered to a nose of the patient and the oxygen treatment is delivered to a mouth of the patient.

In some embodiments, the method further comprises co-administering a secondary therapeutic agent. In some embodiments, the secondary therapeutic agent is an antiviral drug. Non-limiting examples of antiviral drugs include remdesivir, favipiravir, lopinavir/ritonavir, duranavir/cobicistat, and umifenovir (Arbidol). In some embodiments, the secondary therapeutic agent is chloroquine or amodiquine. In some embodiments, the secondary therapeutic agent is bevacizumab. In some embodiments, the secondary therapeutic agent is alpha-interferon. In some embodiments, the secondary therapeutic agent is a corticosteroid (e.g., methylprednisolone, dexamethasone, the like, or a combination thereof). In some embodiments, the secondary therapeutic agent is an antibody. In some embodiments, the secondary therapeutic agent is administered via inhalation. In some embodiments, the secondary therapeutic agent is administered systemically.

In some embodiments, the dose of NO comprises NO and a carrier gas. In some embodiments, the carrier gas comprises a blend of nitric oxide and nitrogen. The blend of nitric oxide and nitrogen may be delivered as INOmax. In some embodiments, the carrier gas is argon. In some embodiments, the concentration of argon is at least 2%. In some embodiments, the carrier gas is helium. Helium may be delivered as Heliox. In some embodiments, the carrier gas is carbon dioxide. In some embodiments, the carrier gas is carbon monoxide. In some embodiments, the carbon monoxide is delivered at a level up to 1000 ppm.

In some embodiments, the method further comprises administering a NO gas transmission facilitator. NO gas transmission facilitators are known in the art and include but are not limited to perfluoron, fluosol, a perfluorinated hydrocarbon, and a pulmonary surfactant. In some embodiments, the pulmonary surfactant is dipalmitoylphosphatidylcholine (DPPC), phosphatidylcholine, a surfactant protein, colfosceril palmitate, pumactant, KL-4, venticute, lucinactant, beractant, calfactant, poractant alfa, or a combination thereof. In some embodiments, the NO gas transmission facilitator is administered via inhalation.

In some embodiments, the method further comprises integrating a NOx scrubbing filter into a gas delivery tube used for delivering the dose of NO.

The present invention provides a method of treating an infection or suspected infection in a patient (e.g., coronavirus infection or suspected coronavirus infection), preventing worsening of symptoms associated with the infection or suspected infection, or ameliorating symptoms associated with the infection or suspected infection. In certain embodiments, the method comprises administering to the patient after exposure or suspected exposure to the coronavirus at least one nitric oxide (NO) treatment, the NO treatment comprising a dose of NO for a time period; followed by administering at least one oxygen treatment, the oxygen treatment comprising a dose of oxygen for a time period. The steps are repeated for at least one cycle. The NO treatment is effective for inhibiting replication of the infectious agent (e.g., coronavirus), preventing worsening of symptoms associated with the infectious agent (e.g., coronavirus), or ameliorating symptoms associated with the infectious agent (e.g., coronavirus). Without wishing to limit the present invention to any theory or mechanism, it is believed that the oxygen treatment is effective for reducing interaction between oxygen and NO that otherwise creates harmful NOx species.

In some embodiments, the patient does not have Severe Acute Respiratory Syndrome (SARS).

In some embodiments, the at least one NO treatment is a single dose of NO. In some embodiments, the at least one NO treatment is two doses of NO. In some embodiments, the at least one NO treatment is a combination of doses of NO.

In some embodiments, the method further comprises measuring metHb levels (SpMet) in a sample from the patient. In some embodiments, the at least one NO treatment is administered such that the SpMet is from 2-20%. In some embodiments, the at least one NO treatment is administered such that the SpMet is from 3-10%. In some embodiments, the at least one NO treatment is administered such that the SpMet is from 5-15%. In some embodiments, the at least one NO treatment is administered such that the SpMet is at least 5%. In some embodiments, SpMet is measured using a non-invasive mechanism, e.g., a pulse CO-oximeter.

In some embodiments, the method further comprises measuring blood nitrate levels in a sample from the patient. In some embodiments, the at least one NO treatment is administered such that the blood nitrate level is from 2,000 ng/mL to 15,000 ng/mL. In some embodiments, the at least one NO treatment is administered such that the blood nitrate level is from 1,400 ng/mL to 15,000 ng/mL. In some embodiments, if the blood nitrate level is over 15,000 ng/mL, then the administration of the at least one NO treatment is stopped.

In some embodiments, the method further comprises administering a compound that increases levels of methemoglobin reductase. In some embodiments, the method further comprises administering an immunosuppressant.

In some embodiments, the method further comprises measuring in the patient one or a combination of: an oxygen saturation level (SpO₂), heart rate, respiration rate, body temperature, blood pressure, an inflammatory cytokine plasma level, a proinflammatory cytokine serum level, a C-reactive protein (CRP) level, a level of lymphocytes, an erythrocyte sedimentation rate (ESR), a procalcitonin (PCT) level, or a serum amyloid A (SAA) protein level.

In some embodiments, the method prevents Severe Acute Respiratory Syndrome (SARS). In some embodiments, the method prevents the patient from having a SpO₂<93% without oxygen supplementation sustained for more than 12 hours. In some embodiments, the method prevents the patient from having a PaO2/FiO2 ratio <300 mmHg sustained for more than 12 hours. In some embodiments, the method prevents the patient from needing high flow nasal cannula oxygen, or intubation and mechanical ventilation, or ECMO therapy over 7 or more days.

In some embodiments, the NO treatment comprises a breath loaded with NO, wherein the NO is at a concentration from 20-500 ppm.

In some embodiments, the breath further comprises oxygen at a concentration from 5-100%. In some embodiments, the breath further comprises oxygen at a concentration from 5-20%. In some embodiments, the oxygen treatment comprises a breath loaded with oxygen, the oxygen is at a concentration from 2-100%. In some embodiments, the oxygen is balanced with N₂, ambient air, another gas, or a combination thereof.

In some embodiments, the NO treatment is delivered to the mouth of the patient, and the oxygen treatment is delivered to the nose of the patient. In some embodiments, the NO treatment is delivered to the nose of the patient, and the oxygen treatment is delivered to the mouth of the patient.

In some embodiments, the method further comprises co-administering a secondary therapeutic agent. In some embodiments, the secondary therapeutic agent is an antiviral drug. Non-limiting examples of antiviral drugs include remdesivir, favipiravir, lopinavir/ritonavir, duranavir/cobicistat, and umifenovir (Arbidol). In some embodiments, the secondary therapeutic agent is chloroquine or amodiquine. In some embodiments, the secondary therapeutic agent is bevacizumab. In some embodiments, the secondary therapeutic agent is alpha-interferon. In some embodiments, the secondary therapeutic agent is a corticosteroid (e.g., methylprednisolone, dexamethasone, the like, or a combination thereof). In some embodiments, the secondary therapeutic agent is an antibody. In some embodiments, the secondary therapeutic agent is administered via inhalation. In some embodiments, the secondary therapeutic agent is administered systemically.

In some embodiments, the dose of NO comprises NO and a carrier gas. In some embodiments, the carrier gas comprises a blend of nitric oxide and nitrogen. The blend of nitric oxide and nitrogen may be delivered as INOmax. In some embodiments, the carrier gas is argon. In some embodiments, the concentration of argon is at least 2%. In some embodiments, the carrier gas is helium. Helium may be delivered as Heliox. In some embodiments, the carrier gas is carbon dioxide. In some embodiments, the carrier gas is carbon monoxide. In some embodiments, the carbon monoxide is delivered at a level up to 1000 ppm.

In some embodiments, the method further comprises administering a NO gas transmission facilitator. NO gas transmission facilitators are known in the art and include but are not limited to perfluoron, fluosol, a perfluorinated hydrocarbon, and a pulmonary surfactant. In some embodiments, the pulmonary surfactant is dipalmitoylphosphatidylcholine (DPPC), phosphatidylcholine, a surfactant protein, colfosceril palmitate, pumactant, KL-4, venticute, lucinactant, beractant, calfactant, poractant alfa, or a combination thereof. In some embodiments, the NO gas transmission facilitator is administered via inhalation.

In some embodiments, the method further comprises integrating a NOx scrubbing filter into a gas delivery tube used for delivering the dose of NO.

For example, the present invention provides a method of treating an infection or suspected infection in a patient (e.g., coronavirus infection or suspected coronavirus infection), preventing worsening of symptoms associated with the infection or suspected infection, or ameliorating symptoms associated with the infection or suspected infection. In certain embodiments, the method comprises administering to the patient after exposure or suspected exposure to the coronavirus at least one nitric oxide (NO) treatment, the NO treatment comprising a dose of 160 ppm NO (or at least 160 ppm NO) for a time period of 30 minutes (e.g., administered over a time period of 30 minutes); followed by administering at least one oxygen treatment, the oxygen treatment comprising a dose of oxygen for/over a time period. The steps are repeated for at least one cycle. In certain embodiments, the method comprises administering to the patient after exposure or suspected exposure to the coronavirus at least one nitric oxide (NO) treatment, the NO treatment comprising a dose of 550 ppm NO for a time period from 5 to 30 minutes (e.g., administered over a time period from 5 to 30 minutes); followed by administering at least one oxygen treatment, the oxygen treatment comprising a dose of oxygen for/over a time period. The steps are repeated for at least one cycle. In certain embodiments, the method comprises administering to the patient after exposure or suspected exposure to the coronavirus at least one nitric oxide (NO) treatment, the NO treatment comprising a dose of 300 ppm NO for a time period from 5 to 45 minutes (e.g., administered over a time period from 5 to 45 minutes); followed by administering at least one oxygen treatment, the oxygen treatment comprising a dose of oxygen for/over a time period. The steps are repeated for at least one cycle. In certain embodiments, the method comprises administering to the patient after exposure or suspected exposure to the coronavirus at least one nitric oxide (NO) treatment, the NO treatment comprising a dose of 100 ppm NO for a time period from 1 to 4 hours (e.g., administered over a time period from 1 to 4 hours); followed by administering at least one oxygen treatment, the oxygen treatment comprising a dose of oxygen for/over a time period. The steps are repeated for at least one cycle. In certain embodiments, the method comprises administering to the patient after exposure or suspected exposure to the coronavirus at least one nitric oxide (NO) treatment, the NO treatment comprising a dose of 80 ppm NO for/over a time period from 1 to 4 hours; followed by administering at least one oxygen treatment, the oxygen treatment comprising a dose of oxygen for/over a time period. The steps are repeated for at least one cycle. In certain embodiments, the method comprises administering to the patient after exposure or suspected exposure to the coronavirus at least one nitric oxide (NO) treatment, the NO treatment comprising a dose of 300 ppm NO for/over a time period of 30 minutes followed by a dose of 80 ppm NO for/over a time period of 2 hours; followed by administering at least one oxygen treatment, the oxygen treatment comprising a dose of oxygen for/over a time period. The steps are repeated for at least one cycle. In certain embodiments, the method comprises administering to the patient after exposure or suspected exposure to the coronavirus at least one nitric oxide (NO) treatment, the NO treatment comprising a dose of 200 ppm NO for/over a time period of 10 minutes followed by a dose of 80 ppm NO for/over a time period of 60 minutes; followed by administering at least one oxygen treatment, the oxygen treatment comprising a dose of oxygen for/over a time period. The steps are repeated for at least one cycle. In certain embodiments, the method comprises administering to the patient after exposure or suspected exposure to the coronavirus at least one nitric oxide (NO) treatment, the NO treatment comprising a dose of 50 ppm NO for/over a time period of at least 5 hours; followed by administering at least one oxygen treatment, the oxygen treatment comprising a dose of oxygen for/over a time period. The steps are repeated for at least one cycle.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows a schematic of an algorithm provided in the present invention. The algorithm may be used to determine whether or not to administer NO. P=one or more parameters being measured (e.g., SpO₂, SpMet, blood pressure (BP), temperature, respiration rate, blood nitrate, etc.); n≥1; T=time.

FIG. 2 shows a schematic of an algorithm provided in the present invention. The algorithm may be used to select a particular treatment (e.g., administration of NO, administration of a drug or other therapeutic composition or therapy, discontinuation of NO, discontinuation of administration of a drug or other therapeutic composition or therapy, etc.). P=one or more parameters being measured (e.g., SpO₂, SpMet, blood pressure (BP), temperature, respiration rate, blood nitrate, etc.); n≥1; T=time.

FIG. 3 shows a schematic of an algorithm provided in the present invention. The algorithm may be used to select a particular treatment (e.g., administration of NO, administration of a drug or other therapeutic composition or therapy, discontinuation of NO, discontinuation of administration of a drug or other therapeutic composition or therapy, etc.) for the purpose of achieving desired values of the parameters (e.g., a desired result). P=one or more parameters being measured (e.g., SpO₂, SpMet, blood pressure (BP), temperature, respiration rate, blood nitrate, etc.); n≥1; T=time.

### DETAILED DESCRIPTION OF THE INVENTION

### Nitric Oxide Administration

As previously discussed, the disclosure provides methods for the treatment of an infection (e.g., respiratory infection) or suspected infection, for the prevention of worsening of symptoms associated with an infection (e.g., respiratory infection) or suspected infection, for ameliorating symptoms associated with an infection (e.g., respiratory infection) or suspected infection, for prevention of an infection (e.g., respiratory infection), for inhibiting proliferation of a virus in a lung of a patient, and for reducing the lethality of an infection (e.g., respiratory infection), for preventing severe acute respiratory syndrome (SARS), etc. The methods herein may be used to treat a patient at any particular stage of infection. However, the present invention generally provides early intervention methods, e.g., methods for treating patients early in the progression of an infection so as to help avoid severe respiratory injury, e.g., to help prevent the development of SARS.

The present disclosure provides specific gaseous nitric oxide (NO) dosing regimens, which may be paired with the monitoring of toxicology outcomes so as to enable the use of high (and effective) NO doses for treatment purposes.

For example, the present invention provides a method of treating a respiratory infection or suspected respiratory infection in a patient. The method comprises administering to the patient after exposure or suspected exposure to the respiratory virus at least one nitric oxide (NO) treatment, the NO treatment comprising a dose of NO for a duration of time, wherein the NO treatment is effective for inhibiting replication of the infectious agent, preventing worsening of symptoms associated with the respiratory infection, or ameliorating symptoms associated with the respiratory infection. The administration of the at least one NO treatment may be repeated as necessary.

Likewise, the present invention also provides a method of preventing worsening of symptoms or ameliorating symptoms associated with a respiratory infection or suspected respiratory infection. The method comprises administering to the patient after exposure or suspected exposure to the respiratory virus at least one nitric oxide (NO) treatment, the NO treatment comprising a dose of NO for a duration of time, wherein the NO treatment is effective for inhibiting replication of the respiratory infection, preventing worsening of symptoms associated with the respiratory infection, or ameliorating symptoms associated with the respiratory infection. The administration of the at least one NO treatment may be repeated as necessary.

Likewise, the present invention also provides a method of preventing severe acute respiratory syndrome (SARS) in a patient with a respiratory infection or suspected respiratory infection. The method comprises administering to the patient after exposure or suspected exposure to the respiratory virus at least one nitric oxide (NO) treatment, the NO treatment comprising a dose of NO for a duration of time, wherein the NO treatment is effective for preventing SARS. The administration of the at least one NO treatment may be repeated as necessary.

The methods herein may help prevent the patient from needing high flow nasal cannula oxygen, or intubation and mechanical ventilation, or ECMO therapy over a certain time period, e.g., 1 or more days, 2 or more days, 3 or more days, 4 or more days, 5 or more days, 6 or more days, 7 or more days, etc.

In certain embodiments, the NO treatment is delivered to a mouth of the patient. In certain embodiments, the NO treatment is delivered to a nose of the patient.

With respect to any of the embodiments herein, the respiratory infection may include but is not limited to infections caused by a coronavirus. Non-limiting examples of human coronaviruses (infectious agents) include SARS-CoV, SARS-CoV-2, MERS-CoV, HCoV-229E, HCoV-NL63, HCoV-OC43, HCoV-HKU1, etc. The present invention is not limited to coronavirus infections such as COVID-19 caused by SARS-CoV-2. As such, the methods herein include methods for treating a coronavirus infection or suspected coronavirus infection in a patient, methods for preventing the worsening of symptoms associated with a coronavirus infection, methods for ameliorating symptoms associated with a coronavirus infection, etc.

In certain embodiments, a patient selected for administering the methods herein is one with the infection or suspected of having the infection, but without SARS.

Respiratory infections may include but are not limited to those caused by an influenza virus, a respiratory syncytial virus (RSV), a parainfluenza virus, a respiratory adenovirus, a rhinovirus, a metapneumonvirus, an enterovirus, etc.

Respiratory infections may include but are not limited to those caused by bacteria such as Streptococcus pneumoniae, Haemophilus influenzae, Moraxella catarrhalis, Staphylococcus aureus, Streptococcus pyogenes etc.

With respect to any of the embodiments herein, the NO treatment may comprise a single concentration of NO for a duration of time, or, a combination of concentrations of NO for combination of durations of time.

Doses of NO may be administered as a time weighted average dose, e.g., a dose of X ppm NO in total administered at a particular rate for a period of time. A non-limiting example is a dose of 72 ppm NO administered at 2 ppb per second for a period of 10 hours.

In certain embodiments, the concentration of NO administered is from 5 ppm to 100 ppm, e.g., 5 ppm, 10 ppm, 20 ppm, 30 ppm, 40 ppm, 50 ppm, 60 ppm, 70 ppm, 80 ppm, 90 ppm, 100 ppm. In certain embodiments, the concentration of NO administered is from 100 ppm to 200 ppm, e.g., 100 ppm, 110 ppm, 120 ppm, 130 ppm, 140 ppm, 150 ppm, 160 ppm, 170 ppm, 180 ppm, 190 ppm, 200 ppm. In certain embodiments, the concentration of NO administered is from 200 ppm to 300 ppm, e.g., 200 ppm, 210 ppm, 220 ppm, 230 ppm, 240 ppm, 250 ppm, 260 ppm, 270 ppm, 280 ppm, 290 ppm, 300 ppm. In certain embodiments, the concentration of NO administered is from 300 ppm to 400 ppm, e.g., 300 ppm, 310 ppm, 320 ppm, 330 ppm, 340 ppm, 350 ppm, 360 ppm, 370 ppm, 380 ppm, 390 ppm, 400 ppm. In certain embodiments, the concentration of NO administered is from 400 ppm to 500 ppm, e.g., 400 ppm, 410 ppm, 420 ppm, 430 ppm, 440 ppm, 450 ppm, 460 ppm, 470 ppm, 480 ppm, 490 ppm, 500 ppm. In certain embodiments, the concentration of NO administered is from 100 ppm to 300 ppm. In certain embodiments, the concentration of NO administered is from 100 ppm to 400 ppm. In certain embodiments, the concentration of NO administered is from 200 ppm to 400 ppm. In certain embodiments, the concentration of NO administered is from 350 ppm to 400 ppm. In certain embodiments, the concentration of NO administered is from 200 ppm to 500 ppm. In certain embodiments, the concentration of NO administered is from 20 ppm to 500 ppm.

In certain embodiments, the duration of time is 1 minute, 2 minutes, 3 minutes, 4 minutes, 5 minutes, 6 minutes, 7 minutes, 8 minutes, 9 minutes, 10 minutes, etc. In certain embodiments, the duration of time is 15 minutes. In certain embodiments, the duration of time is 20 minutes. In certain embodiments, the duration of time is 25 minutes. In certain embodiments, the duration of time is 30 minutes. In certain embodiments, the duration of time is 35 minutes. In certain embodiments, the duration of time is 40 minutes. In certain embodiments, the duration of time is 45 minutes. In certain embodiments, the duration of time is 50 minutes. In certain embodiments, the duration of time is 55 minutes. In certain embodiments, the duration of time is 60 minutes. In certain embodiments, the duration of time is 65 minutes. In certain embodiments, the duration of time is 70 minutes. In certain embodiments, the duration of time is 75 minutes. In certain embodiments, the duration of time is 80 minutes. In certain embodiments, the duration of time is 85 minutes. In certain embodiments, the duration of time is 90 minutes. In certain embodiments, the duration of time is 95 minutes. In certain embodiments, the duration of time is 100 minutes. In certain embodiments, the duration of time is 105 minutes. In certain embodiments, the duration of time is 110 minutes. In certain embodiments, the duration of time is 115 minutes. In certain embodiments, the duration of time is 120 minutes. In certain embodiments, the duration of time is more than 120 minutes. In certain embodiments, the duration of time is 150 minutes. In certain embodiments, the duration of time is 180 minutes. In certain embodiments, the duration of time is more than 180 minutes.

In certain embodiments, the at least one NO treatment is a dose of at least 160 ppm NO for a time period of 30 minutes, e.g., a total of 160 ppm NO administered over a time period of 30 minutes. In certain embodiments, the at least one NO treatment is a dose of 500 ppm NO for a time period from 5 to 30 minutes. In certain embodiments, the at least one NO treatment is a dose of 300 ppm NO for a time period from 5 to 45 minutes. In certain embodiments, the at least one NO treatment is a dose of 100 ppm NO for a time period from 1 to 4 hours. In certain embodiments, the at least one NO treatment is a dose of 80 ppm NO for a time period from 1 to 4 hours. In certain embodiments, the at least one NO treatment is a dose of 300 ppm NO for a time period of 30 minutes followed by a dose of 80 ppm NO for a time period of 2 hours. In certain embodiments, the at least one NO treatment is a dose of 200 ppm NO for a time period of 10 minutes, followed by a dose of 80 ppm NO for a time period of 60 minutes. In certain embodiments, the at least one NO treatment is a dose of 50 ppm NO for a time period of at least 5 hours.

In certain embodiments, the NO treatment comprises more than one concentration of NO, each for a particular duration. For example, in certain embodiments, the NO treatment comprises a first concentration of NO for a first duration of time followed by a second concentration of NO for a second duration of time. In certain embodiments, the NO treatment comprises a first concentration of NO for a first duration of time, followed by a second concentration of NO for a second duration of time, followed by a third concentration of NO for a third duration of time. The first concentration, second concentration, third concentration (or additional concentrations) of NO include any of the concentrations disclosed herein. The first duration, second duration, third duration (or additional durations) include any of the durations disclosed herein.

As a non-limiting example, in certain embodiments, a patient with an infection (e.g., coronavirus infection) (but without SARS) is treated for 30 minutes with a NO concentration of 300 ppm, and then a dose of 80 ppm NO for 2 hours. As a non-limiting example, in certain embodiments, a patient with an infection (e.g., coronavirus infection) (but without SARS) is treated with a dose of 200 ppm NO for 1 minute or less, followed by 80 ppm for 60 minutes.

In certain embodiments, the first concentration of NO is higher than the second concentration of NO. In certain embodiments, the second concentration of NO may be stepped down, e.g., from 80 ppm to 70 ppm, 60 ppm, 50 ppm, 40 ppm, 30 ppm, 20 ppm, 10 ppm, etc.

In certain embodiments, a maintenance dose of NO may be applied. For example, a time weighted average dose of 36 ppm NO in total may be administered at 1 ppb per second for a period of 10 hours. In certain embodiments, a dose of 72 ppm NO may be administered at 2 ppb per second for a period of 10 hours. In certain embodiments, a dose of 180 ppm NO in total may be administered at 5 ppb per second for a period of 10 hours. In certain embodiments, a dose of 360 ppm NO in total may be administered at 10 ppb per second for a period of 10 hours. In other embodiments, the maintenance dose of time weighted average NO ranging from 36 to 360 ppm NO in total is delivered over a period of 4-10 hours.

As previously discussed, in certain embodiments, the at least one NO treatment is repeated. For example, in some embodiments, the at least one NO treatment is repeated 2 or more times over a 24 hour period. In certain embodiments, the at least one NO treatment is repeated 4 or more times over a 24 hour period. In certain embodiments, the at least one NO treatment is repeated 5 or more times over a 24 hour period. In certain embodiments, the at least one NO treatment is repeated 2 or more times over a 2 day period. In certain embodiments, the at least one NO treatment is repeated 2 or more times over a 5 day period. In certain embodiments, the at least one NO treatment is repeated 5 or more times over a 2 day period. In certain embodiments, the at least one NO treatment is repeated 5 or more times over a 5 day period. In certain embodiments, the at least one NO treatment is repeated 25 or more times over a 5 day period.

The NO treatment may comprise NO and a carrier gas. Carrier gasses are well known to one of ordinary skill in the art. For example, in certain embodiments, the carrier gas comprises a blend of nitric oxide and nitrogen, argon, helium, carbon dioxide, carbon monoxide. As a non-limiting example, the blend of nitric oxide and nitrogen may be delivered as INOmax. In certain embodiments, the carrier gas is argon at a concentration of at least 1%. In certain embodiments, the carrier gas is argon at a concentration of at least 2%. In certain embodiments, the carrier gas is argon at a concentration of at least 3%. In certain embodiments, the carrier gas is argon at a concentration of at least 4%. In certain embodiments, the carrier gas is argon at a concentration of at least 5%. In certain embodiments, the carrier gas is helium delivered as Heliox. In certain embodiments, the carrier gas is carbon monoxide delivered at a level up to 100 ppm. In certain embodiments, the carrier gas is carbon monoxide delivered at a level up to 200 ppm. In certain embodiments, the carrier gas is carbon monoxide delivered at a level up to 300 ppm. In certain embodiments, the carrier gas is carbon monoxide delivered at a level up to 400 ppm. In certain embodiments, the carrier gas is carbon monoxide delivered at a level up to 500 ppm. In certain embodiments, the carrier gas is carbon monoxide delivered at a level up to 600 ppm. In certain embodiments, the carrier gas is carbon monoxide delivered at a level up to 700 ppm. In certain embodiments, the carrier gas is carbon monoxide delivered at a level up to 800 ppm. In certain embodiments, the carrier gas is carbon monoxide delivered at a level up to 900 ppm. In certain embodiments, the carrier gas is carbon monoxide delivered at a level up to 1000 ppm.

In certain embodiments, the NO treatment may comprise administering a NO gas transmission facilitator administered via inhalation or other appropriate means. Non-limiting examples of NO gas transmission facilitators include perfluoron, fluosol, a perfluorinated hydrocarbon, or a pulmonary surfactant. Non-limiting examples of a pulmonary surfactant includes dipalmitoylphosphatidylcholine (DPPC), phosphatidylcholine, a surfactant protein, colfosceril palmitate, pumactant, KL-4, venticute, lucinactant, beractant, calfactant, poractant alfa, or a combination thereof.

In certain embodiments, the methods herein further comprise integrating a NOx scrubbing filter into a gas delivery tube used for delivering the dose of NO.

Any appropriate means of delivering the NO treatment may be utilized, and such systems are well known to or would be recognizable to one of ordinary skill in the art. The present invention includes systems or devices for administration of the methods disclosed herein. For example, the present invention includes a ventilation system for performing one or more of the methods disclosed herein.

The methods herein may further comprise measuring metHb levels (SpMet). SpMet may be measured using one or more samples obtained from the patient. In certain embodiments, SpMet is measured using a non-invasive mechanism. For example, SpMet may be measured using a pulse CO-oximeter.

In certain embodiments, SpMet may be monitored to determine if NO administration should be administered, continued, or discontinued. For example, if the SpMet exceeds a threshold of 3%, NO administration may be discontinued. In some embodiments, if the SpMet exceeds a threshold of 5%, NO administration may be discontinued. In some embodiments, if the SpMet exceeds a threshold of 10%, NO administration may be discontinued. In some embodiments, if the SpMet exceeds a threshold of 15%, NO administration may be discontinued. In some embodiments, if the SpMet exceeds a threshold of 20%, NO administration may be discontinued. In some embodiments, if the SpMet exceeds a threshold of 25%, NO administration may be discontinued. In some embodiments, if the SpMet exceeds a threshold of 30%, NO administration may be discontinued.

In certain embodiments, the NO treatment is administered such that the patient's SpMet is from 2-20%. In certain embodiments, the NO treatment is administered such that the patient's SpMet is from 3-10%. In certain embodiments, the NO treatment is administered such that the patient's SpMet is from 5-15%. In certain embodiments, the NO treatment is administered such that the patient's SpMet is at least 5%.

Administration of the NO treatment may be initiated or reinitiated if symptoms worsen or if recommended based on other indicators. For example, if SpMet drops below a certain threshold (e.g., 1%, 2%, 3%, 4%, 5%, 10%, 15%, etc.), the NO treatment may be repeated.

The methods herein may further comprise measuring blood nitrate levels in a sample from the patient. In certain embodiments, the NO treatment is administered such that the patient's blood nitrate level is from 2,000 ng/mL to 15,000 ng/mL. In certain embodiments, the NO treatment is administered such that the patient's blood nitrate level is from 1,400 ng/mL to 15,000 ng/mL. In certain embodiments, blood nitrate levels are used to determine if the administration of the NO treatment should be stopped. For example, if the blood nitrate level is over 15,000 ng/mL, then the administration of the NO treatment is stopped.

The methods herein may further comprise administering a compound that increases levels of methemoglobin reductase.

The methods herein may further comprise measuring in the patient one or a combination of: an oxygen saturation level (SpO₂), heart rate, respiration rate, body temperature, blood pressure, an inflammatory cytokine plasma level, a proinflammatory cytokine serum level (e.g., IL-1b, IFN-gamma, IP-10, or MCP-1), a C-reactive protein (CRP) level, a level of lymphocytes, an erythrocyte sedimentation rate (ESR), a procalcitonin (PCT) level, or a serum amyloid A (SAA) protein level.

In certain embodiments, the methods further comprise administering an immunosuppressant.

In certain embodiments, the methods further comprise co-administering a secondary therapeutic agent. Non-limiting examples of secondary therapeutic agents include antiviral drugs (e.g., remdesivir, favipiravir, lopinavir/ritonavir, duranavir/cobicistat, umifenovir, etc.), an antibody or antibodies (e.g., monoclonal antibodies, antibody cocktails, etc.), chloroquine, amodiquine, bevacizumab, alpha-interferon, a corticosteroid (e.g., dexamethasone, methylprednisolone), the like, combinations thereof, etc. Secondary therapeutic agents may be administered as appropriate, e.g., via inhalation, systemic, etc.

With respect to any of the methods herein, the methods may prevent the patient from having a SpO₂<93% without oxygen supplementation sustained for more than 12 hours.

With respect to any of the methods herein, the methods may prevent the patient from having a PaO2/FiO2 ratio <300 mmHg sustained for more than 12 hours.

In certain embodiments, the infectious agent, e.g., coronavirus, is undetectable 5 days after administration of the at least one NO treatment to the patient. In certain embodiments, the infectious agent, e.g., coronavirus, is undetectable 7 days after administration of the at least one NO treatment to the patient. In certain embodiments, the infectious agent, e.g., coronavirus, is undetectable 10 days after administration of the at least one NO treatment to the patient. In certain embodiments, the infectious agent, e.g., coronavirus, is undetectable 14 days after administration of the at least one NO treatment to the patient. In certain embodiments, the infectious agent, e.g., coronavirus, is undetectable 21 days after administration of the at least one NO treatment to the patient. In certain embodiments, the infectious agent, e.g., coronavirus, is undetectable 28 days after administration of the at least one NO treatment to the patient.

### Nitric Oxide—Oxygen Cycles

As previously discussed, the disclosure provides methods for the treatment of an infection (e.g., respiratory infection) or suspected infection, for the prevention of worsening of symptoms associated with an infection (e.g., respiratory infection) or suspected infection, for ameliorating symptoms associated with an infection (e.g., respiratory infection) or suspected infection, for prevention of an infection (e.g., respiratory infection), for inhibiting proliferation of a virus in a lung of a patient, and for reducing the lethality of an infection (e.g., respiratory infection), for preventing severe acute respiratory syndrome (SARS). The methods herein may be used to treat a patient at any particular stage of infection. However, the present invention provides early intervention methods, e.g., methods for treating patients early in the progression of the infection so as to help avoid severe respiratory injury, e.g., to help prevent the development of SARS.

The present disclosure provides specific gaseous nitric oxide (NO) dosing regimens paired with the monitoring of toxicology outcomes so as to enable the use of high (and effective) NO doses for treatment purposes.

For example, the present invention features a method of treating a respiratory infection or suspected respiratory infection in a patient wherein at least once cycle of a nitric oxide (NO) breath treatment, comprising administering or more concentrations of NO for one or more durations of time, followed by an oxygen breath treatment comprising administering one or more concentrations of oxygen for one or more durations of time, is administered to the patient after exposure or suspected exposure to an infectious agent. The oxygen portion of the method cycle may help to reduce interaction between oxygen and NO that may otherwise create harmful NOx species. The NO breath treatment-oxygen breath treatment cycle helps to inhibit replication of the infectious agent. The NO breath treatment-oxygen breath treatment cycle may be repeated for at least one repetition.

Likewise, the present invention also features a method of preventing worsening of symptoms associated with a respiratory infection or suspected respiratory infection in a patient wherein at least one cycle of a nitric oxide (NO) breath treatment, comprising administering or more concentrations of NO for one or more durations of time, followed by an oxygen breath treatment comprising administering one or more concentrations of oxygen for one or more durations of time, is administered to the patient after exposure or suspected exposure to an infectious agent. The oxygen portion of the method cycle may help to reduce interaction between oxygen and NO that may otherwise create harmful NOx species. The NO breath treatment-oxygen breath treatment cycle helps to prevent worsening of symptoms associated with the respiratory infection. The NO breath treatment-oxygen breath treatment cycle may be repeated for at least one repetition.

Likewise, the present invention also features a method of ameliorating symptoms associated with a respiratory infection or suspected respiratory infection in a patient wherein at least one cycle of a nitric oxide (NO) breath treatment, comprising administering or more concentrations of NO for one or more durations of time, followed by an oxygen breath treatment comprising administering one or more concentrations of oxygen for one or more durations of time, is administered to the patient after exposure or suspected exposure to an infectious agent. The oxygen portion of the method cycle may help to reduce interaction between oxygen and NO that may otherwise create harmful NOx species. The NO breath treatment-oxygen breath treatment cycle helps to ameliorate symptoms associated with the respiratory infection. The NO breath treatment-oxygen breath treatment cycle may be repeated for at least one repetition.

Likewise, the present invention also features a method of preventing severe acute respiratory syndrome (SARS) in a patient with a respiratory infection wherein at least one cycle of a nitric oxide (NO) breath treatment, comprising administering or more concentrations of NO for one or more durations of time, followed by an oxygen breath treatment comprising administering one or more concentrations of oxygen for one or more durations of time, is administered to the patient after exposure or suspected exposure to an infectious agent. The oxygen portion of the method cycle may help to reduce interaction between oxygen and NO that may otherwise create harmful NOx species. The NO breath treatment-oxygen breath treatment cycle helps to prevent SARS. The NO breath treatment-oxygen breath treatment cycle may be repeated for at least one repetition.

In certain embodiments, the NO breath treatment is delivered to a mouth of the patient, and the oxygen breath treatment is delivered to a nose of the patient. In certain embodiments, the NO breath treatment is delivered to a nose of the patient, and the oxygen breath treatment is delivered to a mouth of the patient.

With respect to any of the embodiments herein, the respiratory infection may include but is not limited to infections caused by a coronavirus. Non-limiting examples of human coronaviruses (infectious agents) include SARS-CoV, SARS-CoV-2, MERS-CoV, HCoV-229E, HCoV-NL63, HCoV-OC43, HCoV-HKU1, etc. The present invention is not limited to coronavirus infections such as COVID-19 caused by SARS-CoV-2. As such, the methods herein include methods for treating a coronavirus infection or suspected coronavirus infection in a patient, methods for preventing the worsening of symptoms associated with a coronavirus infection, methods for ameliorating symptoms associated with a coronavirus infection, etc.

In certain embodiments, a patient selected for administering the methods herein is one with the infection or suspected of having the infection, but without SARS.

Respiratory infections may include but are not limited to those caused by an influenza virus, a respiratory syncytial virus (RSV), a parainfluenza virus, a respiratory adenovirus, a rhinovirus, a metapneumonvirus, an enterovirus, etc.

Respiratory infections may include but are not limited to those caused by bacteria such as Streptococcus pneumoniae, Haemophilus influenzae, Moraxella catarrhalis, Staphylococcus aureus, Streptococcus pyogenes etc.

With respect to any of the embodiments herein, the NO breath treatment may comprise a single concentration of NO for a duration of time, or, a combination of concentrations of NO for combination of durations of time.

As previously discussed, doses of NO may be administered as a time weighted average dose, e.g., a dose of X ppm NO in total is administered at a particular rate for a period of time.

In certain embodiments, the concentration of NO administered is from 5 ppm to 100 ppm, e.g., 5 ppm, 10 ppm, 20 ppm, 30 ppm, 40 ppm, 50 ppm, 60 ppm, 70 ppm, 80 ppm, 90 ppm, 100 ppm. In certain embodiments, the concentration of NO administered is from 100 ppm to 200 ppm, e.g., 100 ppm, 110 ppm, 120 ppm, 130 ppm, 140 ppm, 150 ppm, 160 ppm, 170 ppm, 180 ppm, 190 ppm, 200 ppm. In certain embodiments, the concentration of NO administered is from 200 ppm to 300 ppm, e.g., 200 ppm, 210 ppm, 220 ppm, 230 ppm, 240 ppm, 250 ppm, 260 ppm, 270 ppm, 280 ppm, 290 ppm, 300 ppm. In certain embodiments, the concentration of NO administered is from 300 ppm to 400 ppm, e.g., 300 ppm, 310 ppm, 320 ppm, 330 ppm, 340 ppm, 350 ppm, 360 ppm, 370 ppm, 380 ppm, 390 ppm, 400 ppm. In certain embodiments, the concentration of NO administered is from 400 ppm to 500 ppm, e.g., 400 ppm, 410 ppm, 420 ppm, 430 ppm, 440 ppm, 450 ppm, 460 ppm, 470 ppm, 480 ppm, 490 ppm, 500 ppm. In certain embodiments, the concentration of NO administered is from 100 ppm to 300 ppm. In certain embodiments, the concentration of NO administered is from 100 ppm to 400 ppm. In certain embodiments, the concentration of NO administered is from 200 ppm to 400 ppm. In certain embodiments, the concentration of NO administered is from 350 ppm to 400 ppm. In certain embodiments, the concentration of NO administered is from 200 ppm to 500 ppm. In certain embodiments, the concentration of NO administered is from 20 ppm to 500 ppm.

In certain embodiments, the duration of time is 1 minute, 2 minutes, 3 minutes, 4 minutes, 5 minutes, 6 minutes, 7 minutes, 8 minutes, 9 minutes, 10 minutes, etc. In certain embodiments, the duration of time is 15 minutes. In certain embodiments, the duration of time is 20 minutes. In certain embodiments, the duration of time is 25 minutes. In certain embodiments, the duration of time is 30 minutes. In certain embodiments, the duration of time is 35 minutes. In certain embodiments, the duration of time is 40 minutes. In certain embodiments, the duration of time is 45 minutes. In certain embodiments, the duration of time is 50 minutes. In certain embodiments, the duration of time is 55 minutes. In certain embodiments, the duration of time is 60 minutes. In certain embodiments, the duration of time is 65 minutes. In certain embodiments, the duration of time is 70 minutes. In certain embodiments, the duration of time is 75 minutes. In certain embodiments, the duration of time is 80 minutes. In certain embodiments, the duration of time is 85 minutes. In certain embodiments, the duration of time is 90 minutes. In certain embodiments, the duration of time is 95 minutes. In certain embodiments, the duration of time is 100 minutes. In certain embodiments, the duration of time is 105 minutes. In certain embodiments, the duration of time is 110 minutes. In certain embodiments, the duration of time is 115 minutes. In certain embodiments, the duration of time is 120 minutes. In certain embodiments, the duration of time is more than 120 minutes. In certain embodiments, the duration of time is 150 minutes. In certain embodiments, the duration of time is 180 minutes. In certain embodiments, the duration of time is more than 180 minutes.

In certain embodiments, the NO treatment is a dose of at least 160 ppm NO for a time period of 30 minutes, e.g., 160 ppm NO is administered over a time period of 30 minutes. In certain embodiments, the NO treatment is a dose of 500 ppm NO for a time period from 5 to 30 minutes. In certain embodiments, the NO treatment is a dose of 300 ppm NO for a time period from 5 to 45 minutes. In certain embodiments, the NO treatment is a dose of 100 ppm NO for a time period from 1 to 4 hours. In certain embodiments, the NO treatment is a dose of 80 ppm NO for a time period from 1 to 4 hours. In certain embodiments, the NO treatment is a dose of 300 ppm NO for a time period of 30 minutes followed by a dose of 80 ppm NO for a time period of 2 hours. In certain embodiments, the NO treatment is a dose of 200 ppm NO for a time period of 10 minutes, followed by a dose of 80 ppm NO for a time period of 60 minutes. In certain embodiments, the NO treatment is a dose of 50 ppm NO for a time period of at least 5 hours.

In certain embodiments, the NO breath treatment comprises more than one concentration of NO, each for a particular duration. For example, in certain embodiments, the NO breath treatment comprises a first concentration of NO for a first duration of time followed by a second concentration of NO for a second duration of time. In certain embodiments, the NO breath treatment comprises a first concentration of NO for a first duration of time, followed by a second concentration of NO for a second duration of time followed by a third concentration of NO for a third duration of time. The first concentration, second concentration, third concentration (or additional concentrations) of NO include any of the concentrations disclosed herein. The first duration, second duration, third duration (or additional durations) include any of the durations disclosed herein.

As a non-limiting example, in certain embodiments, a patient with an infection (e.g., coronavirus infection) (but without SARS) is treated for 30 minutes with a NO concentration of 300 ppm, and then a dose of 80 ppm NO for 2 hours. As a non-limiting example, in certain embodiments, a patient with an infection (e.g., coronavirus infection) (but without SARS) is treated with a dose of 200 ppm NO for 1 minute or less, followed by 80 ppm for 60 minutes.

In certain embodiments, the first concentration of NO is higher than the second concentration of NO. In certain embodiments, the second concentration of NO may be stepped down, e.g., from 80 ppm to 70 ppm, 60 ppm, 50 ppm, 40 ppm, 30 ppm, 20 ppm, 10 ppm, etc.

In certain embodiments, a maintenance dose of NO may be applied. For example, a time weighted average dose of 36 ppm NO in total may be administered at 1 ppb per second for a period of 10 hours. In certain embodiments, a dose of 72 ppm NO may be administered at 2 ppb per second for a period of 10 hours. In certain embodiments, a dose of 180 ppm NO in total may be administered at 5 ppb per second for a period of 10 hours. In certain embodiments, a dose of 360 ppm NO in total may be administered at 10 ppb per second for a period of 10 hours. In other embodiments, the maintenance dose of time weighted average NO ranging from 36 to 360 ppm NO in total is delivered over a period of 4-10 hours.

As previously discussed, in certain embodiments, the cycle may be repeated. For example, in some embodiments, the cycle is repeated 2 or more times over a 24 hour period. In certain embodiments, the cycle is repeated 4 or more times over a 24 hour period. In certain embodiments, the cycle is repeated 5 or more times over a 24 hour period. In certain embodiments, the cycle is repeated 2 or more times over a 2 day period. In certain embodiments, the cycle is repeated 2 or more times over a 5 day period. In certain embodiments, the cycle is repeated 5 or more times over a 2 day period. In certain embodiments, the cycle is repeated 5 or more times over a 5 day period. In certain embodiments, the cycle is repeated 25 or more times over a 5 day period.

The NO breath treatment may comprise NO and a carrier gas. Carrier gasses are well known to one of ordinary skill in the art. For example, in certain embodiments, the carrier gas comprises a blend of nitric oxide and nitrogen, argon, helium, carbon dioxide, carbon monoxide. As a non-limiting example, the blend of nitric oxide and nitrogen may be delivered as INOmax. In certain embodiments, the carrier gas is argon at a concentration of at least 1%. In certain embodiments, the carrier gas is argon at a concentration of at least 2%. In certain embodiments, the carrier gas is argon at a concentration of at least 3%. In certain embodiments, the carrier gas is argon at a concentration of at least 4%. In certain embodiments, the carrier gas is argon at a concentration of at least 5%. In certain embodiments, the carrier gas is helium delivered as Heliox. In certain embodiments, the carrier gas is carbon monoxide delivered at a level up to 100 ppm. In certain embodiments, the carrier gas is carbon monoxide delivered at a level up to 200 ppm. In certain embodiments, the carrier gas is carbon monoxide delivered at a level up to 300 ppm. In certain embodiments, the carrier gas is carbon monoxide delivered at a level up to 400 ppm. In certain embodiments, the carrier gas is carbon monoxide delivered at a level up to 500 ppm. In certain embodiments, the carrier gas is carbon monoxide delivered at a level up to 600 ppm. In certain embodiments, the carrier gas is carbon monoxide delivered at a level up to 700 ppm. In certain embodiments, the carrier gas is carbon monoxide delivered at a level up to 800 ppm. In certain embodiments, the carrier gas is carbon monoxide delivered at a level up to 900 ppm. In certain embodiments, the carrier gas is carbon monoxide delivered at a level up to 1000 ppm.

In certain embodiments, the NO breath treatment may comprise administering a NO gas transmission facilitator administered via inhalation or other appropriate means. Non-limiting examples of NO gas transmission facilitators include perfluoron, fluosol, a perfluorinated hydrocarbon, or a pulmonary surfactant. Non-limiting examples of a pulmonary surfactant includes dipalmitoylphosphatidylcholine (DPPC), phosphatidylcholine, a surfactant protein, colfosceril palmitate, pumactant, KL-4, venticute, lucinactant, beractant, calfactant, poractant alfa, or a combination thereof.

In certain embodiments, the methods herein further comprise integrating a NOx scrubbing filter into a gas delivery tube used for delivering the dose of NO.

In certain embodiments, the oxygen breath treatment comprises oxygen at a concentration from 2-100%. In certain embodiments, the oxygen breath treatment comprises oxygen at a concentration from 5-100%. In certain embodiments, the oxygen breath treatment comprises oxygen at a concentration from 10-100%. In certain embodiments, the oxygen breath treatment comprises oxygen at a concentration from 20-100%. In certain embodiments, the oxygen breath treatment comprises oxygen at a concentration from 30-100%. In certain embodiments, the oxygen breath treatment comprises oxygen at a concentration from 40-100%. In certain embodiments, the oxygen breath treatment comprises oxygen at a concentration from 50-100%. In certain embodiments, the oxygen breath treatment comprises oxygen at a concentration from 60-100%. In certain embodiments, the oxygen breath treatment comprises oxygen at a concentration from 70-100%. In certain embodiments, the oxygen breath treatment comprises oxygen at a concentration from 80-100%. In certain embodiments, the oxygen breath treatment comprises oxygen at a concentration from 90-100%. In certain embodiments, the oxygen breath treatment comprises oxygen at a concentration from 2-80%, or from 5-80%, or from 10-80%, or from 20-80%, or from 30-80%, or from 40-80%, or from 50-80%, or from 60-80%,or from 70-80%. In certain embodiments, the oxygen breath treatment comprises oxygen at a concentration from 2-60%, or from 5-60%, or from 10-60%, or from 20-60%, or from 30-60%, or from 40-60%, or from 50-60%. In certain embodiments, the oxygen breath treatment comprises oxygen at a concentration from 2-50%, or from 5-50%, or from 10-50%, or from 20-50%, or from 30-50%, or from 40-50%. In certain embodiments, the oxygen breath treatment comprises oxygen at a concentration from 2-40%, or from 5-40%, or from 10-40%, or from 20-40%, or from 30-40%. In certain embodiments, the oxygen breath treatment comprises oxygen at a concentration from 2-20%, or from 5-20%, or from 10-20%.

In certain embodiments, the oxygen breath treatment comprises oxygen at a concentration from 25-30%. In certain embodiments, the oxygen breath treatment comprises oxygen at a concentration from 25-40%. In certain embodiments, the oxygen breath treatment comprises oxygen at a concentration from 25-50%. In certain embodiments, the oxygen breath treatment comprises oxygen at a concentration from 25-60%. In certain embodiments, the oxygen breath treatment comprises oxygen at a concentration from 25-70%. In certain embodiments, the oxygen breath treatment comprises oxygen at a concentration from 25-80%. In certain embodiments, the oxygen breath treatment comprises oxygen at a concentration from 25-90%. In certain embodiments, the oxygen breath treatment comprises oxygen at a concentration from 25-100%. In certain embodiments, the oxygen breath treatment comprises oxygen at a concentration from 30-40%. In certain embodiments, the oxygen breath treatment comprises oxygen at a concentration from 30-50%. In certain embodiments, the oxygen breath treatment comprises oxygen at a concentration from 30-60%. In certain embodiments, the oxygen breath treatment comprises oxygen at a concentration from 30-60%. In certain embodiments, the oxygen breath treatment comprises oxygen at a concentration from 30-60%. In certain embodiments, the oxygen breath treatment comprises oxygen at a concentration from 30-60%. In certain embodiments, the oxygen breath treatment comprises oxygen at a concentration from 30-60%. In certain embodiments, the oxygen breath treatment comprises oxygen at a concentration from 40-50%. In certain embodiments, the oxygen breath treatment comprises oxygen at a concentration from 40-60%. In certain embodiments, the oxygen breath treatment comprises oxygen at a concentration from 40-70%. In certain embodiments, the oxygen breath treatment comprises oxygen at a concentration from 40-80%. In certain embodiments, the oxygen breath treatment comprises oxygen at a concentration from 40-90%. In certain embodiments, the oxygen breath treatment comprises oxygen at a concentration from 40-100%. In certain embodiments, the oxygen breath treatment comprises oxygen at a concentration from 50-60%. In certain embodiments, the oxygen breath treatment comprises oxygen at a concentration from 50-70%. In certain embodiments, the oxygen breath treatment comprises oxygen at a concentration from 50-80%. In certain embodiments, the oxygen breath treatment comprises oxygen at a concentration from 50-90%. In certain embodiments, the oxygen breath treatment comprises oxygen at a concentration from 50-100%. In certain embodiments, the oxygen breath treatment comprises oxygen at a concentration from 60-70%. In certain embodiments, the oxygen breath treatment comprises oxygen at a concentration from 60-80%. In certain embodiments, the oxygen breath treatment comprises oxygen at a concentration from 60-90%. In certain embodiments, the oxygen breath treatment comprises oxygen at a concentration from 60-100%. In certain embodiments, the oxygen breath treatment comprises oxygen at a concentration from 70-80%. In certain embodiments, the oxygen breath treatment comprises oxygen at a concentration from 70-90%. In certain embodiments, the oxygen breath treatment comprises oxygen at a concentration from 70-100%. In certain embodiments, the oxygen breath treatment comprises oxygen at a concentration from 80-90%. In certain embodiments, the oxygen breath treatment comprises oxygen at a concentration from 80-100%. In certain embodiments, the oxygen breath treatment comprises oxygen at a concentration from 90-100%.

In certain embodiments, the duration of oxygen administration is 1 minute, 2 minutes, 3 minutes, 4 minutes, 5 minutes, 6 minutes, 7 minutes, 8 minutes, 9 minutes, 10 minutes, etc. In certain embodiments, the duration of time is 15 minutes. In certain embodiments, the duration of time is 20 minutes. In certain embodiments, the duration of time is 25 minutes. In certain embodiments, the duration of time is 30 minutes. In certain embodiments, the duration of time is 35 minutes. In certain embodiments, the duration of time is 40 minutes. In certain embodiments, the duration of time is 45 minutes. In certain embodiments, the duration of time is 50 minutes. In certain embodiments, the duration of time is 55 minutes. In certain embodiments, the duration of time is 60 minutes. In certain embodiments, the duration of time is 65 minutes. In certain embodiments, the duration of time is 70 minutes. In certain embodiments, the duration of time is 75 minutes. In certain embodiments, the duration of time is 80 minutes. In certain embodiments, the duration of time is 85 minutes. In certain embodiments, the duration of time is 90 minutes. In certain embodiments, the duration of time is 95 minutes. In certain embodiments, the duration of time is 100 minutes. In certain embodiments, the duration of time is 105 minutes. In certain embodiments, the duration of time is 110 minutes. In certain embodiments, the duration of time is 115 minutes. In certain embodiments, the duration of time is 120 minutes. In certain embodiments, the duration of time is more than 120 minutes. In certain embodiments, the duration of time is 150 minutes. In certain embodiments, the duration of time is 180 minutes. In certain embodiments, the duration of time is more than 180 minutes.

As previously discussed, the NO breath treatment-oxygen breath treatment cycle may be repeated for at least one repetition. For example, the NO breath treatment-oxygen breath treatment cycle may be repeated twice. In certain embodiments, the NO breath treatment-oxygen breath treatment cycle may be repeated three times. In certain embodiments, the NO breath treatment-oxygen breath treatment cycle may be repeated three times. In certain embodiments, the NO breath treatment-oxygen breath treatment cycle may be repeated four times. In certain embodiments, the NO breath treatment-oxygen breath treatment cycle may be repeated five times. In certain embodiments, the NO breath treatment-oxygen breath treatment cycle may be repeated six times. In certain embodiments, the NO breath treatment-oxygen breath treatment cycle may be repeated seven times. In certain embodiments, the NO breath treatment-oxygen breath treatment cycle may be repeated eight times. In certain embodiments, the NO breath treatment-oxygen breath treatment cycle may be repeated nine times. In certain embodiments, the NO breath treatment-oxygen breath treatment cycle may be repeated more than 10 times.

In certain embodiments, the oxygen is balanced with N₂, ambient air, another gas, or a combination thereof.

Any appropriate means of delivering the NO breath treatment may be utilized, and such systems are well known to or would be recognizable to one of ordinary skill in the art. The present invention includes systems or devices for administration of the methods disclosed herein. For example, the present invention includes a ventilation system for performing one or more of the methods disclosed herein.

The methods herein may further comprise measuring metHb levels (SpMet). SpMet may be measured using one or more samples obtained from the patient. In certain embodiments, SpMet is measured using a non-invasive mechanism. For example, SpMet may be measured using a pulse CO-oximeter.

In certain embodiments, SpMet may be monitored to determine if NO administration should be administered, continued, or discontinued. For example, if the SpMet exceeds a threshold of 3%, NO administration may be discontinued. In some embodiments, if the SpMet exceeds a threshold of 5%, NO administration may be discontinued. In some embodiments, if the SpMet exceeds a threshold of 10%, NO administration may be discontinued. In some embodiments, if the SpMet exceeds a threshold of 15%, NO administration may be discontinued. In some embodiments, if the SpMet exceeds a threshold of 20%, NO administration may be discontinued. In some embodiments, if the SpMet exceeds a threshold of 25%, NO administration may be discontinued. In some embodiments, if the SpMet exceeds a threshold of 30%, NO administration may be discontinued.

In certain embodiments, the NO breath treatment is administered such that the patient's SpMet is from 2-20%. In certain embodiments, the NO breath treatment is administered such that the patient's SpMet is from 3-10%. In certain embodiments, the NO breath treatment is administered such that the patient's SpMet is from 5-15%. In certain embodiments, the NO breath treatment is administered such that the patient's SpMet is at least 5%.

Administration of the NO breath treatment-oxygen breath treatment cycle may be initiated or reinitiated if symptoms worsen or if recommended based on other indicators. For example, if SpMet drops below a certain threshold (e.g., 1%, 2%, 3%, 4%, 5%, 10%, 15%, etc.), the NO breath treatment-oxygen breath treatment cycle may be repeated.

The methods herein may further comprise measuring blood nitrate levels in a sample from the patient. In certain embodiments, the NO breath treatment-oxygen breath treatment cycle is administered such that the patient's blood nitrate level is from 2,000 ng/mL to 15,000 ng/mL. In certain embodiments, the NO breath treatment-oxygen breath treatment cycle is administered such that the patient's blood nitrate level is from 1,400 ng/mL to 15,000 ng/mL. In certain embodiments, blood nitrate levels are used to determine if the administration of the NO breath treatment-oxygen breath treatment cycle should be stopped. For example, if the blood nitrate level is over 15,000 ng/mL, then the administration of the NO breath treatment-oxygen breath treatment cycle is stopped.

The methods herein may further comprise administering a compound that increases levels of methemoglobin reductase.

The methods herein may further comprise measuring in the patient one or a combination of: an oxygen saturation level (SpO₂), heart rate, respiration rate, body temperature, blood pressure, an inflammatory cytokine plasma level, a proinflammatory cytokine serum level (e.g., IL-1b, IFN-gamma, IP-10, or MCP-1), a C-reactive protein (CRP) level, a level of lymphocytes, an erythrocyte sedimentation rate (ESR), a procalcitonin (PCT) level, or a serum amyloid A (SAA) protein level.

In certain embodiments, the methods further comprise administering an immunosuppressant.

In certain embodiments, the methods further comprise co-administering a secondary therapeutic agent. Non-limiting examples of secondary therapeutic agents include antiviral drugs (e.g., remdesivir, favipiravir, lopinavir/ritonavir, duranavir/cobicistat, umifenovir, etc.), an antibody or antibodies (e.g., monoclonal antibodies, antibody cocktails, etc.), chloroquine, amodiquine, bevacizumab, alpha-interferon, a corticosteroid (e.g., dexamethasone, methylprednisolone), the like, combinations thereof, etc. Secondary therapeutic agents may be administered as appropriate, e.g., via inhalation, systemic, etc.

With respect to any of the methods herein, the methods may prevent the patient from having a SpO₂<93% without oxygen supplementation sustained for more than 12 hours.

With respect to any of the methods herein, the methods may prevent the patient from having a PaO2/FiO2 ratio <300 mmHg sustained for more than 12 hours.

With respect to any of the methods herein, the methods may prevent the patient from needing high flow nasal cannula oxygen, or intubation and mechanical ventilation, or ECMO therapy over 7 or more days.

With respect to any of the methods herein, the methods may prevent the patient from needing high flow nasal cannula oxygen, or intubation and mechanical ventilation, or ECMO therapy over a certain time period, e.g., 1 or more days, 2 or more days, 3 or more days, 4 or more days, 5 or more days, 6 or more days, 7 or more days, etc.

In certain embodiments, the infectious agent, e.g., coronavirus, is undetectable 5 days after administration of the method. In certain embodiments, the infectious agent, e.g., coronavirus, is undetectable 7 days after administration of the method. In certain embodiments, the infectious agent, e.g., coronavirus, is undetectable 10 days after administration of the method. In certain embodiments, the infectious agent, e.g., coronavirus, is undetectable 14 days after administration of the method. In certain embodiments, the infectious agent, e.g., coronavirus, is undetectable 21 days after administration of the method. In certain embodiments, the infectious agent, e.g., coronavirus, is undetectable 28 days after administration of the method.

### Feedback Loop Treatment Methods

As previously discussed, the disclosure provides methods for the treatment of an infection (e.g., respiratory infection) or suspected infection, for the prevention of worsening of symptoms associated with an infection (e.g., respiratory infection) or suspected infection, for ameliorating symptoms associated with an infection (e.g., respiratory infection) or suspected infection, for prevention of an infection (e.g., respiratory infection), for inhibiting proliferation of a virus in a lung of a patient, and for reducing the lethality of an infection (e.g., respiratory infection), for preventing severe acute respiratory syndrome (SARS). The methods herein may be used to treat a patient at any particular stage of infection. However, the present invention provides early intervention methods, e.g., methods for treating patients early in the progression of the infection so as to help avoid severe respiratory injury, e.g., to help prevent the development of SARS.

With respect to any of the embodiments herein, the respiratory infection may include but is not limited to infections caused by a coronavirus. Non-limiting examples of human coronaviruses (infectious agents) include SARS-CoV, SARS-CoV-2, MERS-CoV, HCoV-229E, HCoV-NL63, HCoV-OC43, HCoV-HKU1, etc. The present invention is not limited to coronavirus infections such as COVID-19 caused by SARS-CoV-2. As such, the methods herein include methods for treating a coronavirus infection or suspected coronavirus infection in a patient, methods for preventing the worsening of symptoms associated with a coronavirus infection, methods for ameliorating symptoms associated with a coronavirus infection, etc.

Respiratory infections may include but are not limited to those caused by an influenza virus, a respiratory syncytial virus (RSV), a parainfluenza virus, a respiratory adenovirus, a rhinovirus, a metapneumonvirus, an enterovirus, etc.

Respiratory infections may include but are not limited to those caused by bacteria such as Streptococcus pneumoniae, Haemophilus influenzae, Moraxella catarrhalis, Staphylococcus aureus, Streptococcus pyogenes etc.

In certain embodiments, a patient selected for administering the methods herein is one with the infection or suspected of having the infection, but without SARS.

The present disclosure provides specific gaseous nitric oxide (NO) dosing regimens optionally paired with the monitoring of toxicology outcomes so as to enable the use of effective NO doses for treatment purposes. For example, the present invention features a feedback loop method for treating a respiratory infection, preventing worsening of symptoms of the respiratory infection, ameliorating symptoms of the respiratory infection in a patient in need thereof, or preventing severe acute respiratory syndrome (SARS). The method comprises measuring certain parameters in a patient and using the results of the measurements of the parameters to determine (and administer) an appropriate treatment to administer to the patient in order to achieve desired results of the measurements of the parameters (or variables).

Non-limiting examples of parameters (or variables) that may be measured and/or analyzed include: methemoglobin (metHB) level (SpMet), a blood nitrate level, an oxygen saturation level (SpO₂), heart rate, respiration rate, body temperature, blood pressure, an inflammatory cytokine plasma level, a proinflammatory cytokine serum level, a C-reactive protein (CRP) level, a level of lymphocytes, an erythrocyte sedimentation rate (ESR), a procalcitonin (PCT) level, or a serum amyloid A (SAA) protein level, etc.

The desired results of the measurements of the parameters (or variables) are for treating the respiratory infection, preventing worsening of symptoms thereof, ameliorating symptoms thereof, or preventing SARS.

As an example, the method may comprise determining an observed state of a set of variables in the patient (the set of variables may comprise one or a combination of: a methemoglobin (metHB) level (SpMet), a blood nitrate level, an oxygen saturation level (SpO₂), heart rate, respiration rate, body temperature, blood pressure, an inflammatory cytokine plasma level, a proinflammatory cytokine serum level, a C-reactive protein (CRP) level, a level of lymphocytes, an erythrocyte sedimentation rate (ESR), a procalcitonin (PCT) level, or a serum amyloid A (SAA) protein level). The method may further comprise choosing an appropriate treatment to administer to the patient in order to achieve a target state of the set of variables (the appropriate treatment is chosen based on the observed state of the set of variables. The method may further comprise administering the appropriate treatment decided upon to the patient in order to achieve the target state of the set of variables. The aforementioned steps may be repeated in a loop.

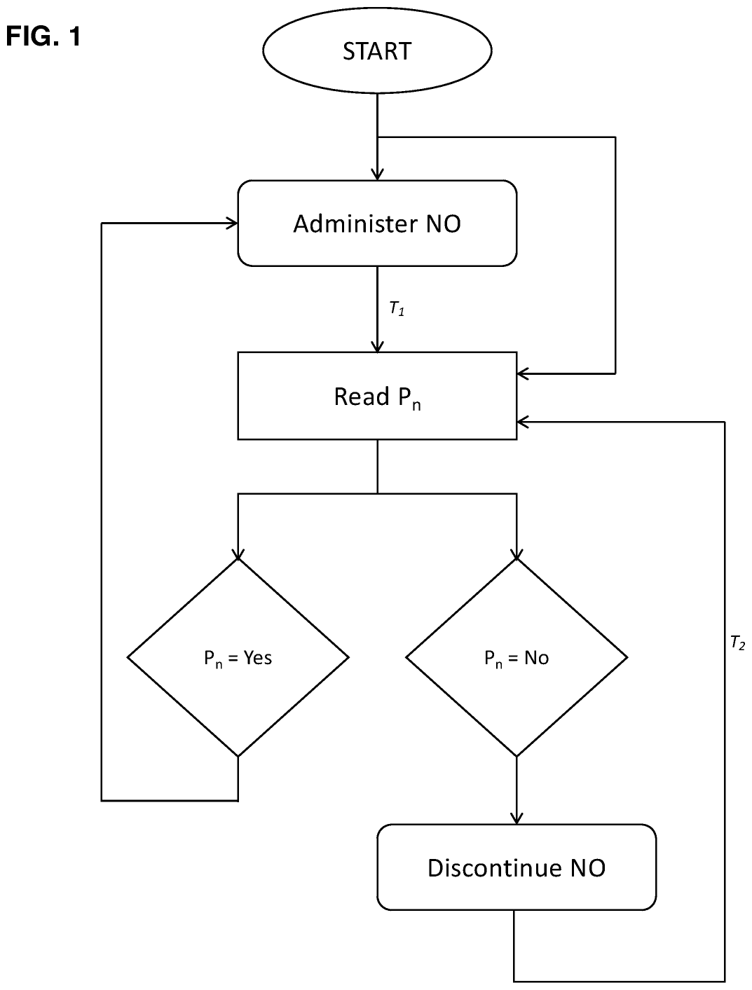

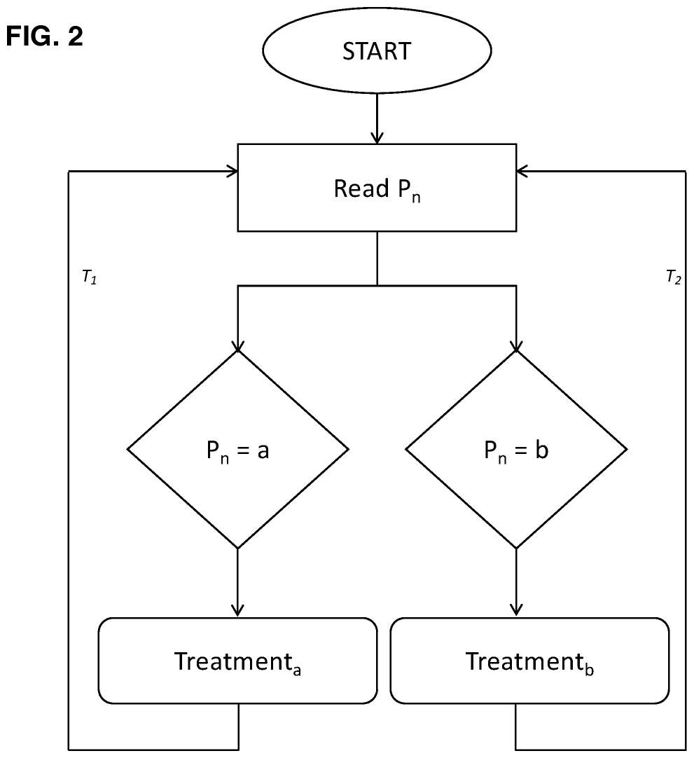

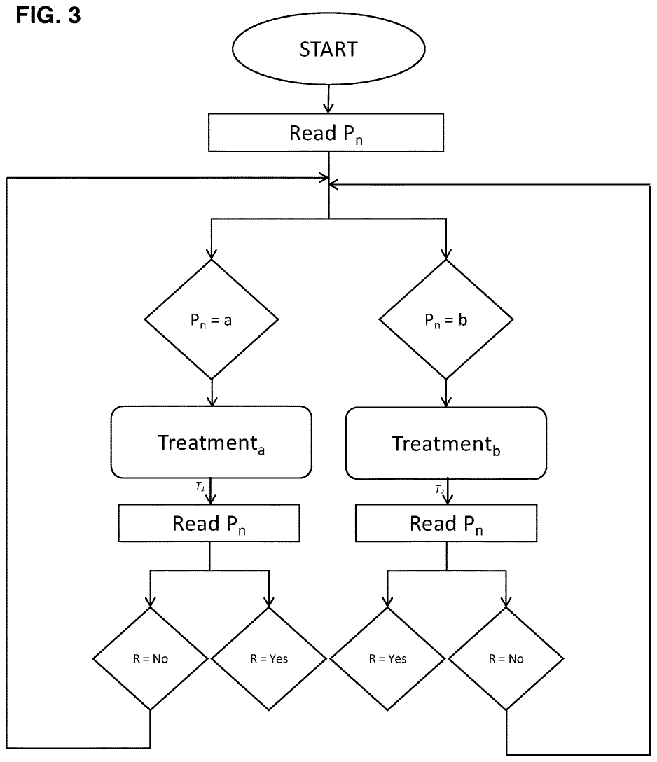

Non-limiting examples of the methods of the present invention are shown schematically in FIG. 1, FIG. 2, and FIG. 3. In FIG. 1, FIG. 2, and FIG. 3, P refers to the one or more parameters being measured (e.g., SpO₂, SpMet, temperature, respiration rate, blood nitrate, heart rate, body temperature, blood pressure, an inflammatory cytokine plasma level, a proinflammatory cytokine serum level, a C-reactive protein (CRP) level, a level of lymphocytes, an erythrocyte sedimentation rate (ESR), a procalcitonin (PCT) level, or a serum amyloid A (SAA) protein level, etc. T refers to a time point (note T₁ may equal T₂, or T₁ may be different from T₂). The number of parameters measured is represented by “n,” wherein n≥1 (e.g., n=1, 2, 3, 4, 5, 6, 7, 8, 9, 10, 11, 12, 13, 14, 15, 16, 17, 18, 19, 20, etc.). For example, in some embodiments, n=2, and the parameters are SpO₂ and SpMet. In some embodiments, n=3, and the parameters are SpO₂, SpMet, and respiration rate. In some embodiments, n=4, and the parameters are SpO₂, SpMet, respiration rate, and heart rate. The present invention is not limited to any particular combination of parameters. Any of the aforementioned parameters may be used in combination with each other though not specifically disclosed.

Referring to FIG. 1, in some embodiments, the method may be used to determine whether or not to administer NO. In certain embodiments, the method begins with administration of NO. In some embodiments, the method begins with the reading of the parameters (e.g., measuring SpO₂, SpMet, temperature, respiration rate, blood nitrate, heart rate, body temperature, blood pressure, etc.). In some embodiments, if the parameters fit a particular formula or criteria (e.g., Pn=yes), the method may comprise initiation of administration of NO or continued administration of NO, whereas if the parameters do not fit the formula or criteria (or fit a different formula or criteria) (e.g., Pn=no), the method may comprise discontinuation of administration of NO.

Referring to FIG. 2, in some embodiments, the method may be used to select a particular treatment, for example, administration of NO, administration of a drug or other therapeutic composition or therapy, discontinuation of administration of NO, discontinuation of administration of a drug or other therapeutic composition or therapy, etc. The method may begin with the reading of the parameters (e.g., measuring SpO₂, SpMet, temperature, respiration rate, blood nitrate, heart rate, body temperature, blood pressure, etc.). In some embodiments, if the parameters fit a particular formula or criteria, e.g., Pn=a, the method may comprise initiation of Treatmenta, In some embodiments, if the parameters fit a particular formula or criteria, e.g., Pn=b, the method may comprise initiation of Treatmentb. In some embodiments, if the parameters fit a particular formula or criteria, e.g., Pn=c, the method may comprise initiation of Treatment. In some embodiments, if the parameters fit a particular formula or criteria (e.g., Pn=d), the method may comprise initiation of Treatmentd. In some embodiments, if the parameters fit a particular formula or criteria (e.g., Pn=e), the method may comprise initiation of Treatmente. The present invention is not limited to Treatmenta, Treatmentb, Treatment, Treatmentd, Treatmente, etc.

Referring to FIG. 3, in some embodiments, the method may be used to select a particular treatment for the purpose of achieving desired values of the parameters (e.g., a desired result). Non-limiting examples of treatments include administration of NO, administration of a drug or other therapeutic composition or therapy, discontinuation of administration of NO, discontinuation of administration of a drug or other therapeutic composition or therapy, etc. The method may begin with the reading of the parameters (e.g., measuring SpO₂, SpMet, temperature, respiration rate, blood nitrate, heart rate, body temperature, blood pressure, etc.). In some embodiments, if the parameters fit a particular formula or criteria, e.g., Pn=a, the method may comprise initiation of Treatmenta, In some embodiments, if the parameters fit a particular formula or criteria, e.g., Pn=b, the method may comprise initiation of Treatmentb. In some embodiments, if the parameters fit a particular formula or criteria, e.g., Pn=c, the method may comprise initiation of Treatmentt. In some embodiments, if the parameters fit a particular formula or criteria (e.g., Pn=d), the method may comprise initiation of Treatmentd. In some embodiments, if the parameters fit a particular formula or criteria (e.g., Pn=e), the method may comprise initiation of Treatmente. The present invention is not limited to Treatmenta, Treatmentb, Treatment, Treatmentd, Treatmente, etc. The treatment may be selected to achieve a particular desired result, e.g., particular measurements or levels of the parameters.

In certain embodiments, the method further comprises reading the parameters again. In some embodiments, if the resulting measurements of the parameters are those that are desired or fit a particular desired formula (e.g., R=Yes), the goal is achieved. In some embodiments, if the resulting measurements of the parameters are not ones that are desired or do not fit a particular desired formula (or fit an alternative formula) (e.g., R=No), then the treatment may be repeated or an alternative treatment may be selected (e.g., depending on the results of the parameters).

In certain embodiments, the appropriate treatment comprises administering gaseous nitric oxide, e.g., at least one NO treatment. In certain embodiments, the NO treatment is delivered to the mouth of the patient. In certain embodiments, the NO treatment is delivered to the nose of the patient.

In certain embodiments, the appropriate treatment comprises a NO treatment-oxygen treatment cycle, e.g., as disclosed herein.

With respect to any of the embodiments herein, the NO treatment may comprise a single concentration of NO for a duration of time, or, a combination of concentrations of NO for combination of durations of time.

As previously discussed, doses of NO may be administered as a time weighted average dose, e.g., a dose of X ppm NO in total is administered at a particular rate for a period of time.

In certain embodiments, the concentration of NO administered is from 5 ppm to 100 ppm, e.g., 5 ppm, 10 ppm, 20 ppm, 30 ppm, 40 ppm, 50 ppm, 60 ppm, 70 ppm, 80 ppm, 90 ppm, 100 ppm. In certain embodiments, the concentration of NO administered is from 100 ppm to 200 ppm, e.g., 100 ppm, 110 ppm, 120 ppm, 130 ppm, 140 ppm, 150 ppm, 160 ppm, 170 ppm, 180 ppm, 190 ppm, 200 ppm. In certain embodiments, the concentration of NO administered is from 200 ppm to 300 ppm, e.g., 200 ppm, 210 ppm, 220 ppm, 230 ppm, 240 ppm, 250 ppm, 260 ppm, 270 ppm, 280 ppm, 290 ppm, 300 ppm. In certain embodiments, the concentration of NO administered is from 300 ppm to 400 ppm, e.g., 300 ppm, 310 ppm, 320 ppm, 330 ppm, 340 ppm, 350 ppm, 360 ppm, 370 ppm, 380 ppm, 390 ppm, 400 ppm. In certain embodiments, the concentration of NO administered is from 400 ppm to 500 ppm, e.g., 400 ppm, 410 ppm, 420 ppm, 430 ppm, 440 ppm, 450 ppm, 460 ppm, 470 ppm, 480 ppm, 490 ppm, 500 ppm. In certain embodiments, the concentration of NO administered is from 100 ppm to 300 ppm. In certain embodiments, the concentration of NO administered is from 100 ppm to 400 ppm. In certain embodiments, the concentration of NO administered is from 200 ppm to 400 ppm. In certain embodiments, the concentration of NO administered is from 350 ppm to 400 ppm. In certain embodiments, the concentration of NO administered is from 200 ppm to 500 ppm. In certain embodiments, the concentration of NO administered is from 20 ppm to 500 ppm.

In certain embodiments, the duration of time is 1 minute, 2 minutes, 3 minutes, 4 minutes, 5 minutes, 6 minutes, 7 minutes, 8 minutes, 9 minutes, 10 minutes, etc. In certain embodiments, the duration of time is 15 minutes. In certain embodiments, the duration of time is 20 minutes. In certain embodiments, the duration of time is 25 minutes. In certain embodiments, the duration of time is 30 minutes. In certain embodiments, the duration of time is 35 minutes. In certain embodiments, the duration of time is 40 minutes. In certain embodiments, the duration of time is 45 minutes. In certain embodiments, the duration of time is 50 minutes. In certain embodiments, the duration of time is 55 minutes. In certain embodiments, the duration of time is 60 minutes. In certain embodiments, the duration of time is 65 minutes. In certain embodiments, the duration of time is 70 minutes. In certain embodiments, the duration of time is 75 minutes. In certain embodiments, the duration of time is 80 minutes. In certain embodiments, the duration of time is 85 minutes. In certain embodiments, the duration of time is 90 minutes. In certain embodiments, the duration of time is 95 minutes. In certain embodiments, the duration of time is 100 minutes. In certain embodiments, the duration of time is 105 minutes. In certain embodiments, the duration of time is 110 minutes. In certain embodiments, the duration of time is 115 minutes. In certain embodiments, the duration of time is 120 minutes. In certain embodiments, the duration of time is more than 120 minutes. In certain embodiments, the duration of time is 150 minutes. In certain embodiments, the duration of time is 180 minutes. In certain embodiments, the duration of time is more than 180 minutes.

In certain embodiments, the at least one NO treatment is a dose of at least 160 ppm NO for a time period of 30 minutes, e.g., a total of 160 ppm NO is administered over a time period of 30 minutes. In certain embodiments, the at least one NO treatment is a dose of 500 ppm NO for a time period from 5 to 30 minutes. In certain embodiments, the at least one NO treatment is a dose of 300 ppm NO for a time period from 5 to 45 minutes. In certain embodiments, the at least one NO treatment is a dose of 100 ppm NO for a time period from 1 to 4 hours. In certain embodiments, the at least one NO treatment is a dose of 80 ppm NO for a time period from 1 to 4 hours. In certain embodiments, the at least one NO treatment is a dose of 300 ppm NO for a time period of 30 minutes followed by a dose of 80 ppm NO for a time period of 2 hours. In certain embodiments, the at least one NO treatment is a dose of 200 ppm NO for a time period of 10 minutes, followed by a dose of 80 ppm NO for a time period of 60 minutes. In certain embodiments, the at least one NO treatment is a dose of 50 ppm NO for a time period of at least 5 hours.

In certain embodiments, the at least one NO treatment comprises a dose of 500 ppm nitric oxide (NO) for a time frame from 5 to 30 minutes. In certain embodiments, the at least one NO treatment comprises a dose of 300 ppm nitric oxide (NO) for a time frame from 5 to 45 minutes. In certain embodiments, the at least one NO treatment comprises a dose of 100 ppm nitric oxide (NO) for a time frame from 1 to 4 hours. In certain embodiments, the at least one NO treatment comprises a dose of 80 ppm nitric oxide (NO) for a time frame from 1 to 4 hours. In certain embodiments, the at least one NO treatment comprises a dose of 300 ppm NO for a time frame of 30 minutes followed by a dose of 80 ppm NO for a time frame of 2 hours. In certain embodiments, the at least one NO treatment comprises a dose of 200 ppm NO for a time frame of 10 minutes, followed by a dose of 80 ppm NO for a time frame of 60 minutes. In certain embodiments, the at least one NO treatment comprises a dose of 50 ppm nitric oxide (NO) for a time frame of at least 5 hours. In certain embodiments, the at least one NO treatment comprises a maintenance dose. In certain embodiments, a maintenance dose of NO may be applied. For example, a time weighted average dose of 36 ppm NO in total may be administered at 1 ppb per second for a period of 10 hours. In certain embodiments, a dose of 72 ppm NO may be administered at 2 ppb per second for a period of 10 hours. In certain embodiments, a dose of 180 ppm NO in total may be administered at 5 ppb per second for a period of 10 hours. In certain embodiments, a dose of 360 ppm NO in total may be administered at 10 ppb per second for a period of 10 hours. In other embodiments the maintenance dose of time weighted average NO ranging from 36 to 360 ppm NO in total is delivered over a period of 4-10 hours.

In certain embodiments, the NO treatment comprises more than one concentration of NO, each for a particular duration. For example, in certain embodiments, the NO treatment comprises a first concentration of NO for a first duration of time followed by a second concentration of NO for a second duration of time. In certain embodiments, the NO treatment comprises a first concentration of NO for a first duration of time followed by a second concentration of NO for a second duration of time followed by a third concentration of NO for a third duration of time. The first concentration, second concentration, third concentration (or additional concentrations) of NO include any of the concentrations disclosed herein. The first duration, second duration, third duration (or additional durations) include any of the durations disclosed herein.

As a non-limiting example, in certain embodiments, a patient with an infection (e.g., coronavirus infection) (but without SARS) is treated for 30 minutes with a NO concentration of 300 ppm, and then a dose of 80 ppm NO for 2 hours. As a non-limiting example, in certain embodiments, a patient with an infection (e.g., coronavirus infection) (but without SARS) is treated with a dose of 200 ppm NO for 1 minute or less, followed by 80 ppm for 60 minutes.

In certain embodiments, the first concentration of NO is higher than the second concentration of NO. In certain embodiments, the second concentration of NO may be stepped down, e.g., from 80 ppm to 70 ppm, 60 ppm, 50 ppm, 40 ppm, 30 ppm, 20 ppm, 10 ppm, etc.

In certain embodiments, a maintenance dose of NO may be applied. For example, a time weighted average dose of 36 ppm NO in total may be administered at 1 ppb per second for a period of 10 hours. In certain embodiments, a dose of 72 ppm NO may be administered at 2 ppb per second for a period of 10 hours. In certain embodiments, a dose of 180 ppm NO in total may be administered at 5 ppb per second for a period of 10 hours. In certain embodiments, a dose of 360 ppm NO in total may be administered at 10 ppb per second for a period of 10 hours. In other embodiments, the maintenance dose of time weighted average NO ranging from 36 to 360 ppm NO in total is delivered over a period of 4-10 hours.

The NO treatment may comprise NO and a carrier gas. Carrier gasses are well known to one of ordinary skill in the art. For example, in certain embodiments, the carrier gas comprises a blend of nitric oxide and nitrogen, argon, helium, carbon dioxide, carbon monoxide. As a non-limiting example, the blend of nitric oxide and nitrogen may be delivered as INOmax. In certain embodiments, the carrier gas is argon at a concentration of at least 1%. In certain embodiments, the carrier gas is argon at a concentration of at least 2%. In certain embodiments, the carrier gas is argon at a concentration of at least 3%. In certain embodiments, the carrier gas is argon at a concentration of at least 4%. In certain embodiments, the carrier gas is argon at a concentration of at least 5%. In certain embodiments, the carrier gas is helium delivered as Heliox. In certain embodiments, the carrier gas is carbon monoxide delivered at a level up to 100 ppm. In certain embodiments, the carrier gas is carbon monoxide delivered at a level up to 200 ppm. In certain embodiments, the carrier gas is carbon monoxide delivered at a level up to 300 ppm. In certain embodiments, the carrier gas is carbon monoxide delivered at a level up to 400 ppm. In certain embodiments, the carrier gas is carbon monoxide delivered at a level up to 500 ppm. In certain embodiments, the carrier gas is carbon monoxide delivered at a level up to 600 ppm. In certain embodiments, the carrier gas is carbon monoxide delivered at a level up to 700 ppm. In certain embodiments, the carrier gas is carbon monoxide delivered at a level up to 800 ppm. In certain embodiments, the carrier gas is carbon monoxide delivered at a level up to 900 ppm. In certain embodiments, the carrier gas is carbon monoxide delivered at a level up to 1000 ppm.

In certain embodiments, the NO treatment may comprise administering a NO gas transmission facilitator administered via inhalation or other appropriate means. Non-limiting examples of NO gas transmission facilitators include perfluoron, fluosol, a perfluorinated hydrocarbon, or a pulmonary surfactant. Non-limiting examples of a pulmonary surfactant include dipalmitoylphosphatidylcholine (DPPC), phosphatidylcholine, a surfactant protein, colfosceril palmitate, pumactant, KL-4, venticute, lucinactant, beractant, calfactant, poractant alfa, or a combination thereof.

In certain embodiments, the methods herein further comprise integrating a NOx scrubbing filter into a gas delivery tube used for delivering the dose of NO.

Any appropriate means of delivering the NO treatment may be utilized, and such systems are well known to or would be recognizable to one of ordinary skill in the art. The present invention includes systems or devices for administration of the methods disclosed herein. For example, the present invention includes a ventilation system for performing one or more of the methods disclosed herein.

In certain embodiments, the method comprises measuring SpMet. SpMet may be measured using one or more samples obtained from the patient. In certain embodiments, SpMet is measured using a non-invasive mechanism. For example, SpMet may be measured using a pulse CO-oximeter.

The method may comprise achieving a target state of the set of variables, wherein the set of variables includes SpMet. For example, in certain embodiments, the target state of the set of variables is a SpMet is from 2-20%. In certain embodiments, the target state of the set of variables is a SpMet is from 3-10%. In certain embodiments, the target state of the set of variables is a SpMet is from 5-15%. In certain embodiments, the target state of the set of variables is a SpMet is of at least 5%.

In certain embodiments, the method comprises measuring blood nitrate level. The method may comprise achieving a target state of the set of variables, wherein the set of variables includes blood nitrate level. For example, in certain embodiments, the target state of the set of variables is a blood nitrate level from 2,000 ng/mL to 15,000 ng/mL. In certain embodiments, the target state of the set of variables is a blood nitrate level from 1,400 ng/mL to 15,000 ng/mL.

In certain embodiments, the method comprises measuring oxygen saturation level (SpO₂). The method may comprise achieving a target state of the set of variables, wherein the set of variables includes an oxygen saturation level (SpO₂).

In certain embodiments, the method comprises measuring heart rate. The method may comprise achieving a target state of the set of variables, wherein the set of variables includes heart rate.

In certain embodiments, the method comprises measuring respiration rate. The method may comprise achieving a target state of the set of variables, wherein the set of variables includes respiration rate.

In certain embodiments, the method comprises measuring body temperature. The method may comprise achieving a target state of the set of variables, wherein the set of variables includes body temperature.

In certain embodiments, the method comprises measuring blood pressure. The method may comprise achieving a target state of the set of variables, wherein the set of variables includes blood pressure.

In certain embodiments, the method comprises measuring an inflammatory cytokine plasma level. The method may comprise achieving a target state of the set of variables, wherein the set of variables includes an inflammatory cytokine plasma level.

In certain embodiments, the method comprises measuring a proinflammatory cytokine serum level (e.g., IL-1b, IFN-gamma, IP-10, MCP-1, etc.). The method may comprise achieving a target state of the set of variables, wherein the set of variables includes a proinflammatory cytokine serum level (e.g., IL-1b, IFN-gamma, IP-10, MCP-1, etc.).

In certain embodiments, the method comprises measuring C-reactive protein (CRP) level. The method may comprise achieving a target state of the set of variables, wherein the set of variables includes C-reactive protein (CRP) level.

In certain embodiments, the method comprises measuring a level of lymphocytes. The method may comprise achieving a target state of the set of variables, wherein the set of variables includes a level of lymphocytes.

In certain embodiments, the method comprises measuring an erythrocyte sedimentation rate (ESR). The method may comprise achieving a target state of the set of variables, wherein the set of variables includes an erythrocyte sedimentation rate (ESR).

In certain embodiments, the method comprises measuring a procalcitonin (PCT) level. The method may comprise achieving a target state of the set of variables, wherein the set of variables includes a procalcitonin (PCT) level.

In certain embodiments, the method comprises measuring a serum amyloid A (SAA) protein level. The method may comprise achieving a target state of the set of variables, wherein the set of variables includes a serum amyloid A (SAA) protein level.

The method may be repeated in a loop for a particular number of repetitions over a period of time. For example, in certain embodiments, the method is repeated in the loop at least 2× in a 24 hour period. In certain embodiments, the method is repeated in the loop at least 4× in a 24 hour period. In certain embodiments, the method is repeated in the loop at least 5× in a 24 hour period.

In certain embodiments, the loop is repeated over a 24 hour period. In certain embodiments, the loop is repeated over a 2 day period. In certain embodiments, the loop is repeated over a 3 day period. In certain embodiments, the loop is repeated over a 5 day period. In certain embodiments, the loop is repeated over a 7 day period. In certain embodiments, the loop is repeated over a 10 day period. In certain embodiments, the loop is repeated over a 14 day period.

The method is not limited to the same treatment for each repetition of the loop. In certain embodiments, the appropriate treatment is different for one or each of the repetitions of the loop. In certain embodiments, the appropriate treatment is the same for each of the repetitions of the loop. In certain embodiments, a portion of the repeats of the loop have the same appropriate treatment, and a portion of the repeats of the loop have a different appropriate treatment.

In certain embodiments, the method further comprises administering a compound that increases levels of methemoglobin reductase.

In certain embodiments, the method further comprises administering an immunosuppressant.

In certain embodiments, the methods further comprise co-administering a secondary therapeutic agent. Non-limiting examples of secondary therapeutic agents include antiviral drugs (e.g., remdesivir, favipiravir, lopinavir/ritonavir, duranavir/cobicistat, umifenovir, etc.), an antibody or antibodies (e.g., monoclonal antibodies, antibody cocktails, etc.), chloroquine, amodiquine, bevacizumab, alpha-interferon, a corticosteroid (e.g., dexamethasone, methylprednisolone), the like, combinations thereof, etc. Secondary therapeutic agents may be administered as appropriate, e.g., via inhalation, systemic, etc.

With respect to any of the methods herein, the methods may prevent the patient from having a SpO₂<93% without oxygen supplementation sustained for more than 12 hours.

With respect to any of the methods herein, the methods may prevent the patient from having a PaO2/FiO2 ratio <300 mmHg sustained for more than 12 hours.

In certain embodiments, the infectious agent, e.g., coronavirus, is undetectable 5 days after administration of the method. In certain embodiments, the infectious agent, e.g., coronavirus, is undetectable 7 days after administration of the method. In certain embodiments, the infectious agent, e.g., coronavirus, is undetectable 10 days after administration of the method. In certain embodiments, the infectious agent, e.g., coronavirus, is undetectable 14 days after administration of the method. In certain embodiments, the infectious agent, e.g., coronavirus, is undetectable 21 days after administration of the method. In certain embodiments, the infectious agent, e.g., coronavirus, is undetectable 28 days after administration of the method.

The methods herein may help prevent the patient from needing high flow nasal cannula oxygen, or intubation and mechanical ventilation, or ECMO therapy over a certain time period, e.g., 1 or more days, 2 or more days, 3 or more days, 4 or more days, 5 or more days, 6 or more days, 7 or more days, etc.

### Air Circulating Systems

In another aspect, the disclosure relates to providing specific gaseous nitric oxide (NO) dosing regimens in air circulating systems, e.g., for the purpose of preventing respiratory infections, for the purpose of reducing spread of respiratory infections, etc.

With respect to any of the embodiments herein, the respiratory infection may include but is not limited to infections caused by a coronavirus. Non-limiting examples of human coronaviruses (infectious agents) include SARS-CoV, SARS-CoV-2, MERS-CoV, HCoV-229E, HCoV-NL63, HCoV-OC43, HCoV-HKU1, etc. The present invention is not limited to coronavirus infections such as COVID-19 caused by SARS-CoV-2.

Respiratory infections may include but are not limited to those caused by an influenza virus, a respiratory syncytial virus (RSV), a parainfluenza virus, a respiratory adenovirus, a rhinovirus, a metapneumonvirus, an enterovirus, etc.

Respiratory infections may include but are not limited to those caused by bacteria such as Streptococcus pneumoniae, Haemophilus influenzae, Moraxella catarrhalis, Staphylococcus aureus, Streptococcus pyogenes etc.

The present disclosure features large capacity air circulating systems, e.g., air circulating systems in spaces equipped to house, shelter, transport, or enclose large numbers of people (e.g., trains, airplanes, buildings such as apartment complexes, offices, shopping locations, etc.), wherein the systems administer nitric oxide (NO).

The present disclosure also features small capacity air circulating systems, e.g., air circulating systems in spaces equipped to house, shelter, transport, or enclose small numbers of people (e.g., passenger cars, trucks, single-family homes, small stores, etc.), wherein the systems administer nitric oxide. A non-limiting example of a small capacity system is a home air purifier.

For example, the system may administer a concentration of NO for a duration of time, and the NO may be administered one or several times per day. For example, the system may repeatedly administer 5 ppm NO for a 5 minute time period each hour.

In some embodiments, the NO concentrations are the same for each repetition. In some embodiments, the durations are the same for each repetition. In some embodiments, the NO concentrations are different for one or each repetition. In some embodiments, the durations are different for one or each repetition.

As previously discussed, doses of NO may be administered as a time weighted average dose, e.g., a dose of X ppm NO in total is administered at a particular rate for a period of time.

In certain embodiments, the concentration of NO administered is from 5 ppm to 100 ppm, e.g., 5 ppm, 10 ppm, 20 ppm, 30 ppm, 40 ppm, 50 ppm, 60 ppm, 70 ppm, 80 ppm, 90 ppm, 100 ppm. In certain embodiments, the concentration of NO administered is from 100 ppm to 200 ppm, e.g., 100 ppm, 110 ppm, 120 ppm, 130 ppm, 140 ppm, 150 ppm, 160 ppm, 170 ppm, 180 ppm, 190 ppm, 200 ppm. In certain embodiments, the concentration of NO administered is from 200 ppm to 300 ppm, e.g., 200 ppm, 210 ppm, 220 ppm, 230 ppm, 240 ppm, 250 ppm, 260 ppm, 270 ppm, 280 ppm, 290 ppm, 300 ppm. In certain embodiments, the concentration of NO administered is from 300 ppm to 400 ppm, e.g., 300 ppm, 310 ppm, 320 ppm, 330 ppm, 340 ppm, 350 ppm, 360 ppm, 370 ppm, 380 ppm, 390 ppm, 400 ppm. In certain embodiments, the concentration of NO administered is from 400 ppm to 500 ppm, e.g., 400 ppm, 410 ppm, 420 ppm, 430 ppm, 440 ppm, 450 ppm, 460 ppm, 470 ppm, 480 ppm, 490 ppm, 500 ppm. In certain embodiments, the concentration of NO administered is from 100 ppm to 300 ppm. In certain embodiments, the concentration of NO administered is from 100 ppm to 400 ppm. In certain embodiments, the concentration of NO administered is from 200 ppm to 400 ppm. In certain embodiments, the concentration of NO administered is from 350 ppm to 400 ppm. In certain embodiments, the concentration of NO administered is from 200 ppm to 500 ppm. In certain embodiments, the concentration of NO administered is from 20 ppm to 500 ppm.

In certain embodiments, the duration of time is 1 minute, 2 minutes, 3 minutes, 4 minutes, 5 minutes, 6 minutes, 7 minutes, 8 minutes, 9 minutes, 10 minutes, etc. In certain embodiments, the duration of time is 15 minutes. In certain embodiments, the duration of time is 20 minutes. In certain embodiments, the duration of time is 25 minutes. In certain embodiments, the duration of time is 30 minutes. In certain embodiments, the duration of time is 35 minutes. In certain embodiments, the duration of time is 40 minutes. In certain embodiments, the duration of time is 45 minutes. In certain embodiments, the duration of time is 50 minutes. In certain embodiments, the duration of time is 55 minutes. In certain embodiments, the duration of time is 60 minutes. In certain embodiments, the duration of time is 65 minutes. In certain embodiments, the duration of time is 70 minutes. In certain embodiments, the duration of time is 75 minutes. In certain embodiments, the duration of time is 80 minutes. In certain embodiments, the duration of time is 85 minutes. In certain embodiments, the duration of time is 90 minutes. In certain embodiments, the duration of time is 95 minutes. In certain embodiments, the duration of time is 100 minutes. In certain embodiments, the duration of time is 105 minutes. In certain embodiments, the duration of time is 110 minutes. In certain embodiments, the duration of time is 115 minutes. In certain embodiments, the duration of time is 120 minutes. In certain embodiments, the duration of time is more than 120 minutes. In certain embodiments, the duration of time is 150 minutes. In certain embodiments, the duration of time is 180 minutes. In certain embodiments, the duration of time is more than 180 minutes.

In some embodiments, the administration of NO is repeated 1 time per hour. In some embodiments, the administration of NO is repeated 2 times per hour. In some embodiments, the administration of NO is repeated 3 times per hour. In some embodiments, the administration of NO is repeated 4 times per hour. In some embodiments, the administration of NO is repeated 5 times per hour. In some embodiments, the administration of NO is repeated 6 times per hour. In some embodiments, the administration of NO is repeated 7 times per hour. In some embodiments, the administration of NO is repeated 8 times per hour. In some embodiments, the administration of NO is repeated 9 times per hour. In some embodiments, the administration of NO is repeated 10 times per hour. In some embodiments, the administration of NO is repeated 11 times per hour. In some embodiments, the administration of NO is repeated 12 times per hour. In some embodiments, the administration of NO is repeated 13 times per hour. In some embodiments, the administration of NO is repeated 14 times per hour. In some embodiments, the administration of NO is repeated 15 times per hour. In some embodiments, the administration of NO is repeated 16 times per hour. In some embodiments, the administration of NO is repeated 17 times per hour. In some embodiments, the administration of NO is repeated 18 times per hour. In some embodiments, the administration of NO is repeated 19 times per hour. In some embodiments, the administration of NO is repeated 20 times per hour. In some embodiments, the administration of NO is repeated over 20 times per hour.

In some embodiments, the administration of NO is repeated 1 time per 12 hours. In some embodiments, the administration of NO is repeated 2 times per 12 hours. In some embodiments, the administration of NO is repeated 3 times per 12 hours. In some embodiments, the administration of NO is repeated 4 times per 12 hours. In some embodiments, the administration of NO is repeated 5 times per 12 hours. In some embodiments, the administration of NO is repeated 6 times per 12 hours. In some embodiments, the administration of NO is repeated 7 times per 12 hours. In some embodiments, the administration of NO is repeated 8 times per 12 hours. In some embodiments, the administration of NO is repeated 9 times per 12 hours. In some embodiments, the administration of NO is repeated 10 times per 12 hours. In some embodiments, the administration of NO is repeated 11 times per 12 hours. In some embodiments, the administration of NO is repeated 12 times per 12 hours. In some embodiments, the administration of NO is repeated 13 times per 12 hours. In some embodiments, the administration of NO is repeated 14 times per 12 hours. In some embodiments, the administration of NO is repeated 15 times per 12 hours. In some embodiments, the administration of NO is repeated 16 times per 12 hours. In some embodiments, the administration of NO is repeated 17 times per 12 hours. In some embodiments, the administration of NO is repeated 18 times per 12 hours. In some embodiments, the administration of NO is repeated 19 times per 12 hours. In some embodiments, the administration of NO is repeated 20 times per 12 hours. In some embodiments, the administration of NO is repeated over 20 times per 12 hours.

In some embodiments, the administration of NO is repeated 1 time per 24 hours. In some embodiments, the administration of NO is repeated 2 times per 24 hours. In some embodiments, the administration of NO is repeated 3 times per 24 hours. In some embodiments, the administration of NO is repeated 4 times per 24 hours. In some embodiments, the administration of NO is repeated 5 times per 24 hours. In some embodiments, the administration of NO is repeated 6 times per 24 hours. In some embodiments, the administration of NO is repeated 7 times per 24 hours. In some embodiments, the administration of NO is repeated 8 times per 24 hours. In some embodiments, the administration of NO is repeated 9 times per 24 hours. In some embodiments, the administration of NO is repeated 10 times per 24 hours. In some embodiments, the administration of NO is repeated 11 times per 24 hours. In some embodiments, the administration of NO is repeated 12 times per 24 hours. In some embodiments, the administration of NO is repeated 13 times per 24 hours. In some embodiments, the administration of NO is repeated 14 times per 24 hours. In some embodiments, the administration of NO is repeated 15 times per 24 hours. In some embodiments, the administration of NO is repeated 16 times per 24 hours. In some embodiments, the administration of NO is repeated 17 times per 24 hours. In some embodiments, the administration of NO is repeated 18 times per 24 hours. In some embodiments, the administration of NO is repeated 19 times per 24 hours. In some embodiments, the administration of NO is repeated 20 times per 24 hours. In some embodiments, the administration of NO is repeated over 20 times per 24 hours.

The NO administration may comprise NO and a carrier gas, as disclosed herein. In certain embodiments, the NO administration may comprise administering a NO gas transmission facilitator, as disclosed herein.

In certain embodiments, the systems also administer oxygen. For example, the system may administer a concentration of oxygen for a duration of time, and the oxygen may be administered one or several times per day. For example, the system may repeatedly administer 20% oxygen for a 5 minute time period each hour. Non-limiting examples of oxygen concentrations and durations are disclosed herein.

In certain embodiments, the systems alternate administering NO and oxygen. In certain embodiments, the systems administer more than one dose of NO before administration of oxygen. The present invention is not limited to any particular number of NO or oxygen doses or combinations thereof.

### EXAMPLE 1

The following is a non-limiting example of the present invention. It is to be understood that said example is not intended to limit the present invention in any way. Equivalents or substitutes are within the scope of the present invention.

NO and O₂ can react and form nitrates or other NOx species that are not therapeutically active and may lead to airway inflammation and damage to lung tissues. The present invention provides methods for minimizing the interaction between O₂ and NO, e.g., so that maximum NO could be delivered to the infected cells.

For example, the methods provide alternating breath cycles, e.g., a NO-loaded breath and followed by an O₂-loaded breath. O₂ concentrations of the O₂-loaded breath may be, for example, from 20-100%, e.g., 20%, 25%, 30%, 35%, 40%, 45%, 50%, 55%, 60%, 65%, 70%, 75%, 80%, 85%, 90%, 95%, 99%, 100% (balanced with N₂ or ambient air, etc.). The NO-loaded breath may be mixed with O₂, e.g., the NO may be mixed with oxygen, e.g., 0-100%, e.g., 5%, 10%, 15%, 20%, etc.

Alternating breath cycles may involve repeated cycles of one breath of NO followed by one breath of O₂. However, the present invention also encompasses variations thereof, such as repeated cycles of one breath of NO followed by two breaths of O₂, two breaths of NO followed by one breath of O₂, one breath of NO followed by three breaths of O₂, one breath of O₂ followed by two breaths of NO, two breaths of O₂ followed by one breath of NO, one breath of O₂ followed by three breaths of NO, etc.

The patient may be equipped with separate NO and O₂ lines, e.g., one O₂ line to the nose and one NO line to the mouth, or vice versa. As an example, the NO line to the mouth may feature NO (e.g., 100-300 ppm) with a low level of oxygen (e.g., 20%), and the oxygen line to the nose may feature an oxygen concentration of 80%. Table 1 below shows non-limiting examples of embodiments of the present invention, wherein a particular gas mixture is delivered to the nose, and a particular gas mixture is delivered to the mouth. Note that the gas mixture that is listed as being delivered to the nose may be switched with that listed as being delivered to the mouth (the present invention is not limited to the routes of delivery).

TABLE 1

| | Nose | | |
|---|---|---|---|
| | O₂ | Mouth | |
| | (balanced with | NO | |
| Embodiment | ambient air or N2) | (ppm) | O₂ |
| 1 | 20% | 200 | 0% |
| 2 | 40% | 50 | 50% |
| 3 | 60% | 100 | 10% |
| 4 | 80% | 500 | 5% |
| 5 | 60% | 300 | 20% |
| 6 | 40% | 20 | 10% |
| 7 | 80% | 300 | 5% |
| 8 | 100% | 400 | 20% |
| 9 | 20% | 100 | 50% |
| 10 | 30% | 500 | 5% |

Mechanisms for mechanical switching of inhalation gas are known to one of ordinary skill in the art. For example, mechanisms for switching of ventilation may be achieved with assist control (AC), synchronized intermittent mandatory ventilation (SIMV), etc.

The present invention also provides the use of different carrier gasses.

INOMAX (nitric oxide gas) is a drug administered by inhalation. The INOMAX formulation is a gaseous blend of nitric oxide and nitrogen (0.08% and 99.92%, respectively for 800 ppm). Gaseous NO is supplied in aluminum cylinders as a compressed gas under high pressure (2000 pounds per square inch gauge [psig]). Exemplary devices for blending NO gas with room air and or oxygen, for example the INOBLENDER allows users to select a concentration of NO gas to be mixed into a user set flow of oxygen which is delivered to a patient. When the device is used with an oxygen/air blender, specifications can be set to deliver NO with 100% oxygen. In particular, the use of 100% oxygen at 3.4 bar (50 psig) is the labeled specification for the INOBLENDER device. A user may determine that some clinical conditions may necessitate the use of an oxygen/air blender with the device to achieve FiO2 levels less than 100%. Using oxygen/air mixtures (21% to 95% v/v) will reduce the delivered NO concentration by up to 10% of setting or 1 ppm (whichever is greater) compared to using 100% oxygen alone, resulting in a cumulative error up to +/−30% of setting or 3 ppm (whichever is greater).

The present invention describes the use of Argon as a larger constituent of the makeup gas compared to N₂. Ambient air comprises about 1% Ar. The methods herein feature the use of higher levels of Ar, e.g., 2% Ar, which may help decrease NO to NOx conversions by oxygen.

Another carrier gas may include helium (see, for example, Gentile, M., 2011 Respiratory Care 56:1341-1359). Heliox (a mixture of helium and oxygen) has been reported to be effective in a variety of respiratory conditions such as upper-airway obstruction, status asthmaticus, decompression sickness, post-extubation stridor, bronchiolitis, and ARDS.

reducing the work of breathing (WOB). Heliox converts areas of extreme turbulence and makes these areas less turbulent. Additionally, Heliox converts some areas of turbulence to areas of more efficient laminar flow. Thus, Heliox improves the efficiency of gas flow through narrowed orifices.

Other carrier gasses may include carbon dioxide (see Curosurf below) and low levels of Carbon Monoxide (e.g., up to 1000 ppm)

The present invention also includes NOx scrubbing. NOx scrubbing features a filter that scrubs the NOx species out of the gas line just before being inhaled by the patient. Non-limiting examples of NOx scrubbing compositions include alkali salts like KOH pellets. The scrubbing compositions and methods may be incorporated as an in-line device added to the gas delivery tube.

The present invention also provides methods for enhancing the uptake of NO. Because NO is lipophilic, entry into solution is not optimal for biological systems. The present invention provides methods for enhancing solubility/entry of NO into the cells, for example by combining NO with a nebulized treatment of a NO gas transmission facilitator.

Non-limiting examples of NO gas transmission facilitators (that area administered via inhalation) include perfluron, fluosol, perfluorinated hydrocarbons, pulmonary surfactants (e.g., lipoprotein complexes formed by type II alveolar cells, dipalmitoylphosphatidylcholine (DPPC), phosphatidylcholine, surfactant proteins (SP-A, SP-B, SP-C, SP-D), colfosceril palmitate, pumactant, KL-4, venticute, lucinactant, beractant, calfactant, poractant alfa (Curosurf), etc.), etc. For example, nebulized perflubon is a synthetic surfactant having mucolytic properties. In certain embodiments, the perflubron-based formulation may be emulsified with lecithin (e.g., as the only surfactant). In certain embodiments, the perflubron-based formulation has a median particle diameter of ˜0.17 pm. Fluosol is an oxygen-carrying emulsion comprising two perfluorochemicals (perfluorodecalin and perfluorotripropylamine).

The present invention also includes combination therapies, wherein NO gas is combined with one or a combination of other therapeutic agents (e.g., antiviral agent, etc.) or therapeutic treatments. Agents or treatments may be administered via any appropriate mechanism, such as local administration via inhalation, systemic administration, etc. Non-limiting examples of other therapeutic agents that may be combined with NO gas includes: antiviral drugs (e.g., remdesivir, favipiravir, lopinavir/ritonavir, duranavir/cobicistat, umifenovir, etc.), an antibody or antibodies (e.g., monoclonal antibodies, antibody cocktails, etc.), chloroquine, amodiquine, bevacizumab, alpha-interferon, a corticosteroid (e.g., dexamethasone, methylprednisolone), the like, combinations thereof, etc. Secondary therapeutic agents may be administered as appropriate, e.g., via inhalation, systemic, etc.

### EXAMPLE 2

The following is a non-limiting example of proposed clinical endpoints for a clinical trial. It is to be understood that said example is not intended to limit the present invention in any way. Equivalents or substitutes are within the scope of the present invention.

Clinical trials related to the methods of the present invention may utilize one or a combination of the following clinical endpoints:

(1) Resolution of infection, e.g., virus undetectable at a particular time point. In certain embodiments, the time point is 7 days. In certain embodiments, the time point is 10 days. In certain embodiments, the time point is 12 days. In certain embodiments, the time point is 14 days. In certain embodiments, the time point is 20 days. In certain embodiments, the time point is 21 days. In certain embodiments, the time point is 24 days. In certain embodiments, the time point is 28 days.

(2) Reduction in the proportion of early stage patients who progress to a severe form of the disease. In certain embodiments, the severe form of the disease is defined as SpO₂<93% without oxygen supplementation sustained for more than 12 hours. In certain embodiments, the severe form of the disease is defined as PaO2/FiO2 ratio <300 mmHg sustained for more than 12 hours. In certain embodiments, the severe form of the disease is defined as the necessity of high flow nasal cannula oxygen or intubation and mechanical ventilation or ECMO therapy over 7 days. In certain embodiments, the severe form of the disease is defined as the necessity of high flow nasal cannula oxygen or intubation and mechanical ventilation or ECMO therapy over 14 days. In certain embodiments, the severe form of the disease is defined as the necessity of high flow nasal cannula oxygen or intubation and mechanical ventilation or ECMO therapy over 28 days. The present invention is not limited to the aforementioned definitions of the severe form of the disease.

(3) Percentage of patients developing worsening symptoms, e.g., at a particular time point. In certain embodiments, the time point is daily over the course of a certain number of days. In certain embodiments, the time point is 2 days. In certain embodiments, the time point is 3 days. In certain embodiments, the time point is 4 days. In certain embodiments, the time point is 3 days. In certain embodiments, the time point is 5 days. In certain embodiments, the time point is 3 days. In certain embodiments, the time point is 6 days. In certain embodiments, the time point is 3 days. In certain embodiments, the time point is 7 days. In certain embodiments, the time point is 3 days. In certain embodiments, the time point is 10 days. In certain embodiments, the time point is 3 days. In certain embodiments, the time point is 12 days. In certain embodiments, the time point is 3 days. In certain embodiments, the time point is 14 days.

(4) Overall survival at a particular time point. In certain embodiments, the time point is 7 days. In certain embodiments, the time point is 8 days. In certain embodiments, the time point is 10 days. In certain embodiments, the time point is 12 days. In certain embodiments, the time point is 14 days. In certain embodiments, the time point is 20 days. In certain embodiments, the time point is 21 days. In certain embodiments, the time point is 24 days. In certain embodiments, the time point is 28 days. In certain embodiments, the time point is 30 days. In certain embodiments, the time point is 60 days. In certain embodiments, the time point is 90 days.

The following documents are each incorporated herein by reference in their entirety: U.S. Pat. No. 10,039,781, U.S. Pat. Application No. 2007/0116785, U.S. Pat. Application No. 2010/0040703, U.S. Pat. Nos. 8,043,252, 7,955,294, 10,335,567, U.S. Pat. Application No. 2015/0272988, U.S. Pat. No. 10,039,781, U.S. Pat. Application No. 2019/0091425, U.S. Pat. Nos. 5,558,083, 5,558,083, 5,558,083, 5,732,693, 5,752,504, 6,125,846, 7,114,510, 8,282,966, 8,291,904, 8,291,904, 8,293,284, 8,431,163, 8,573,209, 8,573,210, 6,164,276, 6,109,260, 8,518,457, 8,083,997, 8,079,998, 8,066,904, 8,057,742, 7,531,133, 7,516,742, 6,432,077, 7,516,742, 7,955,294, U.S. Patent Application No. 2011/0262335, U.S. Pat. Application No. 2011/0259325, U.S. Pat. Application No. 2011/0240019, U.S. Pat. Application No. 2011/0220103, U.S. Pat. Application No. 2010/0331405, U.S. Pat. Application No. 2011/0112468, U.S. Pat. Application No. 2008/0287861, U.S. Pat. Application No. 2008/0193566, U.S. Pat. Application No. 2007/0116785, U.S. Pat. Application No. 2007/0104653, U.S. Pat. Application No. 2007/0088316, U.S. Pat. Application No. 2007/0086954, U.S. Pat. Application No. 2007/0065473, U.S. Pat. Application No. 2007/0014688, U.S. Pat. Application No. 2006/0207594, U.S. Pat. Application No. 2005/0191372, WO 1995/10315, WO 2008/095312, WO 2006/071957, WO 2006/110923, WO 2006/110923, WO 2007/057763, WO 2007/057763, WO 2000/30659, and EP 0692984.

Embodiments of the present invention can be freely combined with each other if they are not mutually exclusive.

As used herein, the term “and/or” includes any and all combinations of one or more of the associated listed items.

As used herein, the singular forms “a,” “an,” and “the” are intended to include the plural forms as well, unless the context clearly indicates otherwise. It will be further understood that the terms “comprises,” “comprising,” “includes,” and/or “including” when used herein specify the presence of stated features, integers, steps, operations, elements, and/or components, but do not preclude the presence or addition of one or more other features, integers, steps, operations, elements, components, and/or groups thereof.

Unless stated otherwise, terms used herein have the same meaning as commonly understood by one of ordinary skill in the art.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. A feedback loop method for at least one of: treating a coronavirus infection, preventing worsening of symptoms of the coronavirus infection, and ameliorating symptoms of the coronavirus infection in a patient in need thereof, said method comprising:
a) determining an observed state of a set of variables in the patient, wherein the set of variables comprises one or a combination of: a methemoglobin (metHB) level (SpMet), a blood nitrate level, an oxygen saturation level (SpO₂), heart rate, respiration rate, body temperature, blood pressure, an inflammatory cytokine plasma level, a proinflammatory cytokine serum level, a C-reactive protein (CRP) level, a level of lymphocytes, an erythrocyte sedimentation rate (ESR), a procalcitonin (PCT) level, or a serum amyloid A (SAA) protein level;
b) choosing an appropriate treatment to administer to the patient in order to achieve a target state of the set of variables, the appropriate treatment chosen based on the observed state of the set of variables in (a); and
c) administering the appropriate treatment decided upon in (b) to the patient in order to achieve the target state of the set of variables;
wherein the method continuously repeats steps (a), (b), and (c) in order in a loop, wherein the target state of the set of variables is for at least one of: treating a coronavirus infection, preventing worsening of symptoms thereof, and ameliorating symptoms thereof.

2. The method of claim 1, wherein the appropriate treatment comprises administering to the patient at least one nitric oxide (NO) treatment, the NO treatment comprising a dose of NO for a time period.

3. The method of claim 2, wherein the appropriate treatment further comprises administering at least one oxygen treatment subsequent to the at least one NO treatment, the oxygen treatment comprising a dose of oxygen for a time period, the oxygen treatment is essentially free of NO.

4. The method of claim 1, wherein the appropriate treatment comprises administering to the patient at least one nitric oxide (NO) treatment, the NO treatment comprising a dose of NO for a time period, and after completion of the at least one NO treatment subsequently administering at least one oxygen treatment, the oxygen treatment comprising a dose of oxygen for a time period, the oxygen treatment is essentially free of NO.

5. A method of at least one of: treating a coronavirus infection or suspected coronavirus infection in a patient, preventing worsening of symptoms associated with the coronavirus infection or suspected coronavirus infection, and ameliorating symptoms associated with the coronavirus infection or suspected coronavirus infection, said method comprising:
a. administering to the patient after exposure or suspected exposure to the coronavirus at least one nitric oxide (NO) treatment, the NO treatment comprising a dose of NO for a time period; followed by
b. administering at least one oxygen treatment after cessation of the at least one NO treatment, the oxygen treatment comprising a dose of oxygen for a time period, the oxygen treatment is essentially free of NO;
wherein steps (a) and (b) are performed as one cycle or repeated for at least one additional cycle, wherein the oxygen treatment is effective for reducing interaction between oxygen and NO that otherwise creates harmful NOx species;
wherein the NO treatment is effective for at least one of: inhibiting replication of the coronavirus, preventing worsening of symptoms associated with the coronavirus, and ameliorating symptoms associated with the coronavirus.

6. The method of claim 5, wherein the method further comprises, before step (a), after step (a), after step (b), or a combination thereof, (i) determining an observed state of a set of variables in the patient, the set of variables comprises one or a combination of: a methemoglobin (metHB) level (SpMet), a blood nitrate level, an oxygen saturation level (SpO₂), heart rate, respiration rate, body temperature, blood pressure, an inflammatory cytokine plasma level, a proinflammatory cytokine serum level, a C-reactive protein (CRP) level, a level of lymphocytes, an erythrocyte sedimentation rate (ESR), a procalcitonin (PCT) level, or a serum amyloid A (SAA) protein level; (ii) choosing an appropriate treatment to administer to the patient in order to achieve a target state of the set of variables, the appropriate treatment is chosen based on the observed state of the set of variables in (i); and (iii) administering the appropriate treatment decided upon in (ii) to the patient in order to achieve the target state of the set of variables; wherein the method continuously repeats (i), (ii), and (iii) in order in a loop, wherein the target state of the set of variables is for treating a coronavirus infection, preventing worsening of symptoms thereof, or ameliorating symptoms thereof.

7. The method of claim 6, wherein the appropriate treatment comprises administering to the patient at least one nitric oxide (NO) treatment, the NO treatment comprising a dose of NO for a time period.

8. The method of claim 7, wherein the appropriate treatment further comprises administering at least one oxygen treatment subsequent to the at least one NO treatment, the oxygen treatment comprising a dose of oxygen for a time period, the oxygen treatment is essentially free of NO.

9. The method of claim 6, wherein the appropriate treatment comprises administering to the patient at least one nitric oxide (NO) treatment, the NO treatment comprising a dose of NO for a time period, and after completion of the at least one NO treatment subsequently administering at least one oxygen treatment, the oxygen treatment comprising a dose of oxygen for a time period, the oxygen treatment is essentially free of NO.
